(19) United States
(12) Patent Application Publication
(10) Pub. No.: US 2005/0009841 A1
(43) Pub. Date: Jan. 13, 2005

(54) Novel amides useful for treating pain

(76) Inventors: Guo Zhu Zheng, Lake Bluff, IL (US); Brian S. Brown, Evanston, IL (US); Sean C. Turner, Mannheim (DE); Tammie K. White, Gurnee, IL (US); Robert G. Schmidt, Waukegan, IL (US); John R. Koenig, Chicago, IL (US); Chih-Hung Lee, Vernon Hills, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/618,016

(22) Filed: Jul. 11, 2003

(51) Int. Cl. A61K 31/505; A61K 31/44; A61K 31/4172

(52) U.S. Cl. 514/256; 514/357; 514/400; 544/335; 546/336; 548/335.5

(57) ABSTRACT

The present invention relates to compounds of formula (I) 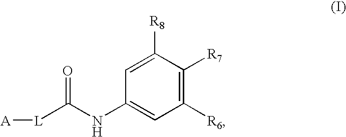 that useful in treating pain.

### TECHNICAL BACKGROUND

The present invention relates to compounds of formula (I-IV) that are useful for treating pain and pharmaceutical compositions containing compounds of formula (I-IV) that are useful in treating pain.

### BACKGROUND OF INVENTION

Pain continues to produce severe distress in people's lives, dominating and disrupting their quality of life. Much of the currently available clinical treatment is only partially effective and may be accompanied by distressing side effects or have abuse potential. The unmet clinical need, the personal suffering, and societal economic costs of pain are substantial. The lack of success in clinical pain therapy exemplifies the need for the discovery of new analgesics.

The present invention relates to novel compounds useful as analgesics.

### SUMMARY OF THE PRESENT INVENTION

The present invention discloses novel amides, a method for controlling pain in mammals, and pharmaceutical compositions including those amides. More particularly, the present invention is directed to compounds of formula (I) 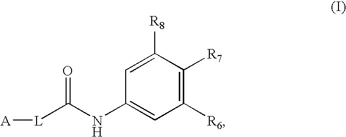
or a pharmaceutically acceptable salt or prodrug thereof, wherein

A is

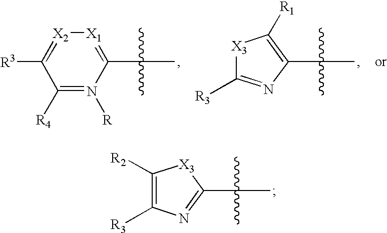

X₁ is N or CR₁;

X₂ is N or CR₂;

X₃ is O or S;

R is absent or O;

R₁ is hydrogen, lower alkoxy, lower alkenyl, lower alkyl, lower alkylthio, lower alkynyl, lower haloalkoxy, lower haloalkyl, lower haloalkylthio, halogen, hydroxy, mercapto, nitro, or —NR_{A}R_{B};

R₂, R₃, and R₄ are independently hydrogen or halogen;

R₇ is hydrogen, alkenyl, alkoxy, alkoxycarbonyl, alkoxysulfonyl, alkyl, alkylcarbonyl, alkylsulfonyl, alkylthio, alkynyl, aryl, arylalkyl, aryloxy, arylthio, cycloalkyl, cycloalkylalkyl, cycloalkyloxy, cycloalkylthio, haloalkoxy, haloalkyl, haloalkylsulfonyl, haloalkylthio, halogen, heteroaryl, heteroarylalkyl, heteroaryloxy, heteroarylthio, heterocycle, heterocyclealkyl, hydroxy, hydroxyalkyl, —NR_{C}R_{D}, (NR_{A}R_{B})carbonyl, or (NR_{A}R_{B})sulfonyl;

R₆ and R₈ are independently hydrogen, lower alkenyl, lower alkoxy, lower alkyl, lower alkylthio, lower alkynyl, lower haloalkoxy, lower haloalkyl, lower haloalkylthio, halogen, hydroxy, mercapto, or —NR_{A}R_{B};

R_{A} and R_{B} are independently hydrogen or alkyl;

R_{C} and R_{D} are independently hydrogen, alkenyl, alkoxycarbonyl, alkyl, alkylcarbonyl, alkynyl, or (NR_{A}R_{B})carbonyl;

L is

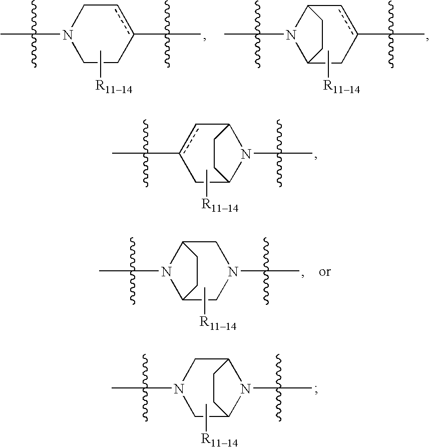

- - - is absent or a single bond; and

R₁₁, R₁₂, R₁₃, and R₁₄ are independently hydrogen, alkoxy, alkyl, or hydroxy.

### DETAILED DESCRIPTION OF THE PRESENT INVENTION

In the principle embodiment, compounds of formula (I) are disclosed 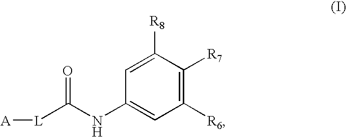
or a pharmaceutically acceptable salt or prodrug thereof, wherein

A is

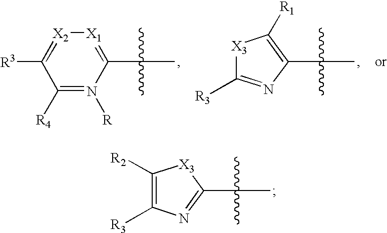

X₁ is N or CR₁;

X₂ is N or CR₂;

X₃ is O or N;

R is absent or O;

R₁ is hydrogen, lower alkoxy, lower alkenyl, lower alkyl, lower alkylthio, lower alkynyl, lower haloalkoxy, lower haloalkyl, lower haloalkylthio, halogen, hydroxy, mercapto, nitro, or —NR_{A}R_{B};

R₂, R₃; and R₄ are independently hydrogen or halogen;

R₇ is hydrogen, alkenyl, alkoxy, alkoxycarbonyl, alkoxysulfonyl, alkyl, alkylcarbonyl, alkylsulfonyl, alkylthio, alkynyl, aryl, arylalkyl, aryloxy, arylthio, cycloalkyl, cycloalkylalkyl, cycloalkyloxy, cycloalkylthio, haloalkoxy, haloalkyl, haloalkylsulfonyl, haloalkylthio, halogen, heteroaryl, heteroarylalkyl, heteroaryloxy, heteroarylthio, heterocycle, heterocyclealkyl, hydroxy, hydroxyalkyl, —NR_{C}R_{D}, (NR_{A}R_{B})carbonyl, or (NR_{A}R_{B})sulfonyl;

R₆ and R₈ are independently hydrogen, lower alkenyl, lower alkoxy, lower alkyl, lower alkylthio, lower alkynyl, lower haloalkoxy, lower haloalkyl, lower haloalkylthio, halogen, hydroxy, mercapto, or —NR_{A}R_{B};

R_{A} and R_{B} are independently hydrogen or alkyl;

R_{C} and R_{D} are independently hydrogen, alkenyl, alkoxycarbonyl, alkyl, alkylcarbonyl, alkynyl, or (NR_{A}R_{B})carbonyl;

L is

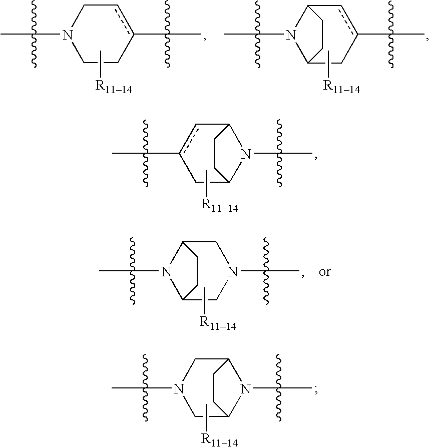

- - - is absent or a single bond; and

R₁₁, R₁₂, R₁₃, and R₁₄ are independently hydrogen, alkoxy, alkyl, or hydroxy.

Another embodiment of the present invention relates to compounds of formula (II) 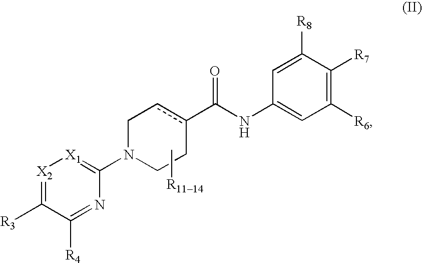
or a pharmaceutically acceptable salt or prodrug thereof wherein - - - is absent or a single bond; X₁ is N or CR₁; X₂ is N or CR₂; R₁ is hydrogen, lower alkoxy, lower alkenyl, lower alkyl, lower alkylthio, lower alkynyl, lower haloalkoxy, lower haloalkyl, lower haloalkylthio, halogen, hydroxy, mercapto, nitro, or —NR_{A}R_{B}; R₂, R₃, and R₄ are independently hydrogen or halogen; R₇ is hydrogen, alkenyl, alkoxy, alkoxycarbonyl, alkoxysulfonyl, alkyl, alkylcarbonyl, alkylsulfonyl, alkylthio, alkynyl, aryl, arylalkyl, aryloxy, arylthio, cycloalkyl, cycloalkylalkyl, cycloalkyloxy, cycloalkylthio, haloalkoxy, haloalkyl, haloalkylsulfonyl, haloalkylthio, halogen, heteroaryl, heteroarylalkyl, heteroaryloxy, heteroarylthio, heterocycle, heterocyclealkyl, hydroxy, hydroxyalkyl, —NR_{C}R_{D}, (NR_{A}R_{B})carbonyl, or (NR_{A}R_{B})sulfonyl; R₆ and R₈ are independently hydrogen, lower alkenyl, lower alkoxy, lower alkyl, lower alkylthio, lower alkynyl, lower haloalkoxy, lower haloalkyl, lower haloalkylthio, halogen, hydroxy, mercapto, or —NR_{A}R_{B}; R_{A} and R_{B} are independently hydrogen or alkyl; R_{C} and R_{D} are independently hydrogen, alkenyl, alkoxycarbonyl, alkyl, alkylcarbonyl, alkynyl, or (NR_{A}R_{B})carbonyl; and R₁₁, R₁₂, R₁₃, and R₁₄ are independently hydrogen, alkoxy, alkyl, or hydroxy.

Another embodiment of the present invention relates to compounds of formula (II) wherein - - - is a single bond; X₁ is CR₁; X₂ is CR₂; R₁ is lower haloalkyl or halogen; R₂, R₃, and R₄ are hydrogen; R₇ is alkoxy, alkyl, alkylthio, haloalkoxy, haloalkylsulfonyl, haloalkyl, haloalkylthio, halogen, or —NR_{C}R_{D}; R₁₁, R₁₂, R₁₃, and R₁₄ are hydrogen; R_{C} and R_{D} are independently hydrogen or alkyl; and R₆ and R₈ are as defined in formula (II).

Another embodiment of the present invention relates to compounds of formula (II) wherein - - - is a single bond; X₁ is CR₁; X₂ is CR₂; R₁ is lower haloalkyl or halogen wherein the halogen or haloalkyl is —Cl or trifluoromethyl; R₂, R₃, and R₄ are hydrogen; R₆ is hydrogen, lower alkyl, lower haloalkyl, or halogen; R₇ is alkoxy, alkyl, alkylthio, haloalkoxy, haloalkylsulfonyl, haloalkyl, haloalkylthio, halogen, or —NR_{C}R_{D}; R₈, R₁₁, R₁₂, R₁₃, and R₁₄ are hydrogen; and R_{C} and R_{D} are independently hydrogen or alkyl.

Another embodiment of the present invention relates to compounds of formula (II) wherein - - - is a single bond; X₁ is CR₁; X₂ is CR₂; R₁ is lower haloalkyl or halogen wherein the halogen or haloalkyl is —Cl or trifluoromethyl; R₂, R₃, and R₄ are hydrogen; R₇ is halogen wherein the halogen is —Cl; and R₆, R₈, R₁₁, R₁₂, R₁₃, and R₁₄ are hydrogen.

Another embodiment of the present invention relates to compounds of formula (II) wherein - - - is a single bond; X₁ is CR₁; X₂ is CR₂; R₁ is lower haloalkyl or halogen; R₂, R₃, and R₄ are hydrogen; R₇ is aryl or aryloxy; R₁₁, R₁₂, R₁₃, and R₁₄ are hydrogen; and R₆, and R₈ are as defined in formula (II).

Another embodiment of the present invention relates to compounds of formula (II) wherein - - - is a single bond; X₁ is CR₁; X₂ is CR₂; R₁ is lower haloalkyl or halogen wherein the halogen or haloalkyl is —Cl or trifluoromethyl; R₂, R₃, and R₄ are hydrogen; R₆ is hydrogen, lower alkyl, lower haloalkyl, or halogen; R₇ is aryl wherein the aryl is phenyl; and R₈, R₁₁, R₁₂, R₁₃, and R₁₄ are hydrogen.

Another embodiment of the present invention relates to compounds of formula (II) wherein - - - is a single bond; X₁ is CR₁; X₂ is CR₂; R₁ is lower haloalkyl or halogen wherein the halogen or haloalkyl is —Cl or trifluoromethyl; R₂, R₃, and R₄ are hydrogen; R₆ is hydrogen, lower alkyl, lower haloalkyl, or halogen; R₇ is aryloxy wherein the aryl of the aryloxy is phenyl; and R₈, R₁₁, R₁₂, R₁₃, and R₁₄ are hydrogen.

Another embodiment of the present invention relates to compounds of formula (II) wherein wherein - - - is a single bond; X₁ is CR₁; X₂ is CR₂; R₁ is lower haloalkyl or halogen; R₂, R₃, and R₄ are hydrogen; R₇ is heterocycle; R₁₁, R₁₂, R₁₃, and R₁₄ are hydrogen; and R₆, and R₈ are as defined in formula (II).

Another embodiment of the present invention relates to compounds of formula (II) wherein wherein - - - is a single bond; X₁ is CR₁; X₂ is CR₂; R₁ is lower haloalkyl or halogen wherein the halogen or haloalkyl is —Cl or trifluoromethyl; R₂, R₃, and R₄ are hydrogen; R₆ is hydrogen, lower alkyl, lower haloalkyl, or halogen; R₇ is heterocycle wherein the heterocycle is azepanyl, morpholinyl, or piperidinyl; and R₈, R₁₁, R₁₂, R₁₃, and R₁₄ are hydrogen.

Another embodiment of the present invention relates to compounds of formula (II) wherein wherein - - - is a single bond; X₁ is CR₁; X₂ is CR₂; R₁ is lower haloalkyl or halogen wherein the halogen or haloalkyl is —Cl or trifluoromnethyl; R₂, R₃, and R₄ are hydrogen; R₆ is lower alkyl, lower haloalkyl, or halogen; and R₇, R₈, R₁₁, R₁₂, R₁₃, and R₁₄ are hydrogen.

Another embodiment of the present invention relates to compounds of formula (II) wherein wherein - - - is a single bond; X₁ is CR₁; X₂ is CR₂; R₁ is lower haloalkyl or halogen wherein the halogen or haloalkyl is —Cl or trifluoromethyl; R₂, R₃, and R₄ are hydrogen; R₆ is lower alkyl, lower haloalkyl, or halogen; and R₇, R₈, R₁₁, R₁₂, R₁₃, and R₁₄ are hydrogen.

Another embodiment of the present invention relates to compounds of formula (II) wherein - - - is absent; X₁ is CR₁; X₂ is CR₂; R₁ is lower haloalkyl or halogen; R₂, R₃, and R₄ are hydrogen; R₇ is alkoxy, alkyl, alkylthio, haloalkoxy, haloalkylsulfonyl, haloalkyl, halogen, or —NR_{C}R_{D}; R₁₁ is hydrogen or hydroxy; R₁₂, R₁₃, and R₁₄ are hydrogen; R_{C} and R_{D} are independently hydrogen or alkyl; and R₆ and R₈ are as defined in formula (II).

Another embodiment of the present invention relates to compounds of formula (II) wherein - - - is absent; X₁ is CR₁; X₂ is CR₂; R₁ is lower haloalkyl or halogen wherein the halogen or haloalkyl is —Cl or trifluoromethyl; R₂, R₃, and R₄ are hydrogen; R₆ is hydrogen, lower alkyl, lower haloalkyl, or halogen; R₇ is alkoxy, alkyl, alkylthio, haloalkoxy, haloalkyl, haloalkylsulfonyl, halogen, or —NR_{C}R_{D}; R₁₁ is hydroxy; R₈, R₁₂, R₁₃, and R₁₄ are hydrogen; and R_{C} and R_{D} are independently hydrogen or alkyl.

Another embodiment of the present invention relates to compounds of formula (II) wherein - - - is a single bond; X₁ is N; X₂ is CR₂; R₂, R₃, and R₄ are hydrogen; R₇ is alkoxy, alkyl, alkylthio, haloalkoxy, haloalkyl, haloalkylsulfonyl, haloalkylthio, halogen, or —NR_{C}R_{D}; R₁₁, R₁₂, R₁₃, and R₁₄ are hydrogen; R_{C} and R_{D} are independently hydrogen or alkyl; and R₆ and R₈ are as defined in formula (II).

Another embodiment of the present invention relates to compounds of formula (II) wherein - - - is a single bond; X₁ is N; X₂ is CR₂; R₂, R₃, R₄, and R₈ are hydrogen; R₆ is hydrogen, lower alkyl, lower haloalkyl, or halogen; R₇ is alkoxy, alkyl, alkylthio, haloalkoxy, haloalkyl, haloalkylsulfonyl, haloalkylthio, halogen, or —NR_{C}R_{D}; R₁₁, R₁₂, R₁₃, and R₁₄ are hydrogen; and R_{C} and R_{D} are independently hydrogen or alkyl.

Another embodiment of the present invention relates to compounds of formula (III) 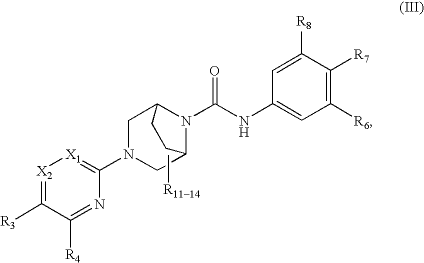
or a pharmaceutically acceptable salt or prodrug thereof wherein X₁ is N or CR₁; X₂ is N or CR₂; R₁ is hydrogen, lower alkoxy, lower alkenyl, lower alkyl, lower alkylthio, lower alkynyl, lower haloalkoxy, lower haloalkyl, lower haloalkylthio, halogen, hydroxy, mercapto, nitro, or —NR_{A}R_{B}; R₂, R₃, and R₄ are independently hydrogen or halogen; R₇ is hydrogen, alkenyl, alkoxy, alkoxycarbonyl, alkoxysulfonyl, alkyl, alkylcarbonyl, alkylsulfonyl, alkylthio, alkynyl, aryl, arylalkyl, aryloxy, arylthio, cycloalkyl, cycloalkylalkyl, cycloalkyloxy, cycloalkylthio, haloalkoxy, haloalkyl, haloalkylsulfonyl, haloalkylthio, halogen, heteroaryl, heteroarylalkyl, heteroaryloxy, heteroarylthio, heterocycle, heterocyclealkyl, hydroxy, hydroxyalkyl, —NR_{C}R_{D}, (NR_{A}R_{B})carbonyl, or (NR_{A}R_{B})sulfonyl; R₆ and R₈ are independently hydrogen, lower alkenyl, lower alkoxy, lower alkyl, lower alkylthio, lower alkynyl, lower haloalkoxy, lower haloalkyl, lower haloalkylthio, halogen, hydroxy, mercapto, or —NR_{A}R_{B}; R_{A} and R_{B} are independently hydrogen or alkyl; and R_{C} and R_{D} are independently hydrogen, alkenyl, alkoxycarbonyl, alkyl, alkylcarbonyl, alkynyl, or (NR_{A}R_{B})carbonyl.

Another embodiment of the present invention relates to compounds of formula (III) wherein X₁ is CR₁; X₂ is CR₂; R₁ is lower haloalkyl or halogen; R₂, R₃, and R₄ are hydrogen; R₇ is alkoxy, alkyl, alkylthio, haloalkoxy, haloalkyl, haloalkylthio, halogen, or —NR_{C}R_{D}; R₁₁, R₁₂, R₁₃, and R₁₄ are hydrogen; R_{C} and R_{D} are independently hydrogen or alkyl; and R₆ and R₈ are as defined in formula (III).

Another embodiment of the present invention relates to compounds of formula (III) wherein X₁ is CR₁; X₂ is CR₂; R₁ is lower haloalkyl or halogen wherein the halogen or haloalkyl is —Cl or trifluoromethyl; R₂, R₃, and R₄ are hydrogen; R₆ is hydrogen, lower alkyl, lower haloalkyl, or halogen; R₇ is alkoxy, alkyl, alkylthio, haloalkoxy, haloalkyl, haloalkylthio, halogen, or —NR_{C}R_{D}; R₈, R₁₁, R₁₂, R₁₃, and R₁₄ are hydrogen; and R_{C} and R_{D} are independently hydrogen or alkyl.

Another embodiment of the present invention relates to compounds of formula (III) wherein X₁ is CR₁; X₂ is CR₂; R₁ is lower haloalkyl or halogen wherein the halogen or haloalkyl is —Cl or trifluromethyl; R₂, R₃, and R₄ are hydrogen; R₆ is lower alkyl, lower haloalkyl, or halogen; and R₇, R₈, R₁₁, R₁₂, R₁₃, and R₁₄ are hydrogen.

Another embodiment of the present invention relates to compounds of formula (III) wherein X₁ is CR₁; X₂ is CR₂; R₁ is lower haloalkyl or halogen wherein the halogen or haloalkyl is —Cl or trifluromethyl; R₂, R₃, and R₄ are hydrogen; R₆ is lower alkyl, lower haloalkyl, or halogen; and R₇, R₈, R₁₁, R₁₂, R₁₃, and R₁₄ are hydrogen.

Another embodiment of the present invention relates to compounds of formula (IV) 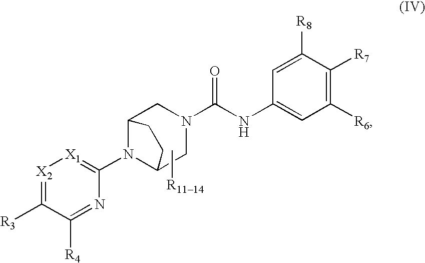
or a pharmaceutically acceptable salt or prodrug wherein X₁ is N or CR₁; X₂ is N or CR₂; R₁ is hydrogen, lower alkoxy, lower alkenyl, lower alkyl, lower alkylthio, lower alkynyl, lower haloalkoxy, lower haloalkyl, lower haloalkylthio, halogen, hydroxy, mercapto, nitro, or —NR_{A}R_{B}; R₂, R₃, and R₄ are independently hydrogen or halogen; R₇ is hydrogen, alkenyl, alkoxy, alkoxycarbonyl, alkoxysulfonyl, alkyl, alkylcarbonyl, alkylsulfonyl, alkylthio, alkynyl, aryl, arylalkyl, aryloxy, arylthio, cycloalkyl, cycloalkylalkyl, cycloalkyloxy, cycloalkylthio, haloalkoxy, haloalkyl, haloalkylsulfonyl, haloalkylthio, halogen, heteroaryl, heteroarylalkyl, heteroaryloxy, heteroarylthio, heterocycle, heterocyclealkyl, hydroxy, hydroxyalkyl, —NR_{C}R_{D}, (NR_{A}R_{B})carbonyl, or (NR_{A}R_{B})sulfonyl; R₆ and R₈ are independently hydrogen, lower alkenyl, lower alkoxy, lower alkyl, lower alkylthio, lower alkynyl, lower haloalkoxy, lower haloalkyl, lower haloalkylthio, halogen, hydroxy, mercapto, or —NR_{A}R_{B}; R_{A} and R_{B} are independently hydrogen or alkyl; and R_{C} and R_{D} are independently hydrogen, alkenyl, alkoxycarbonyl, alkyl, alkylcarbonyl, alkynyl, or (NR_{A}R_{B})carbonyl.

Another embodiment of the present invention relates to compounds of formula (IV) wherein X₁ is CR₁; X₂ is CR₂; R₁ is lower haloalkyl or halogen; R₂, R₃, and R₄ are hydrogen; R₇ is alkoxy, alkyl, alkylthio, haloalkoxy, haloalkyl, haloalkylthio, halogen, or —NR_{C}R_{D}; R₁₁, R₁₂, R₁₃, and R₁₄ are hydrogen; R_{C} and R_{D} are independently hydrogen or alkyl; and R₆ and R₈ are as defined in formula (IV).

Another embodiment of the present invention relates to compounds of formula (IV) wherein X₁ is CR₁; X₂ is CR₂; R₁ is lower haloalkyl or halogen wherein the halogen or haloalkyl is —Cl or trifluoromethyl; R₂, R₃, and R₄ are hydrogen; R₇ is alkoxy, alkyl, alkylthio, haloalkoxy, haloalkyl, haloalkylthio, halogen, or —NR_{C}R_{D}; R₆, R₈, R₁₁, R₁₂, R₁₃, and R₁₄ are hydrogen; and R_{C} and R_{D} are independently hydrogen or alkyl.

Another embodiment of the present invention relates to pharmaceutical compositions comprising a therapeutically effective amount of a compound of formula (I-IV) or a pharmaceutically acceptable salt thereof.

Another embodiment of the present invention relates to a method for treating pain in a manmmal, comprising administering a therapeutically effective amount of a compound of formula (I-IV) or a pharmaceutically acceptable salt thereof.

Definition of Terms

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxysulfonyl” as used herein, means an alkoxy group, as defined herein, appended appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” as used herein, means a monocyclic-ring system, a bicyclic-fused ring system, or a tricyclic-fused ring system wherein one or more of the fused rings are aromatic. Representative examples of aryl include, but are not limited to, anthracenyl, azulenyl, fluorenyl, 2,3-dihydroindenyl, indenyl, naphthyl, phenyl, and tetrahydronaphthyl.

The aryl groups of this invention can be optionally substituted with 1, 2, 3, or 4 substituents independently selected from alkenyl, alkoxy, alkoxyalkyl, alkoxycarbonyl, alkoxysulfonyl, alkyl, alkylcarbonyl, alkylcarbonyloxy, alkylsulfonyl, alkylthio, alkynyl, carboxy, cyano, formyl, haloalkoxy, haloalkyl, haloalkylsulfonyl, haloalkylthio, halogen, hydroxy, hydroxyalkyl, mercapto, nitro, —NR_{A}R_{B}, (NR_{A}R_{B})carbonyl, and (NR_{A}R_{B})sulfonyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “aryloxy” as used herein, means an aryl group, as defined herein, appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “arylthio” as used herein, means an aryl group, as defined herein, appended to the parent molecular moiety through a sulfur atom. Representative examples of arylthio include, but are not limited to, phenylthio and 2-naphthylthio.

The term “carbonyl” as used herein, means a —C(O)— group.

The term “carboxy” as used herein, means a —CO₂H group.

The term “cyano” as used herein, means a —CN group.

The term “cycloalkyl” as used herein, means a saturated cyclic hydrocarbon group containing from 3 to 8 carbons. Examples of cycloalkyl include cyclopropyl, cyclobutyl, cyclopentyl, cyclohexyl, cycloheptyl, and cyclooctyl.

The cycoalkyl groups of the present invention are optionally substituted with 1, 2, 3, or 4 substituents selected from alkenyl, alkoxy, alkoxyalkyl, alkoxycarbonyl, alkoxysulfonyl, alkyl, alkylcarbonyl, alkylcarbonyloxy, alkylsulfonyl, alkylthio, alkynyl, carboxy, cyano, formyl, haloalkoxy, haloalkyl, haloalkylsulfonyl, haloalkylthio, halogen, hydroxy, hydroxyalkyl, mercapto, —NR_{A}R_{B}, (NR_{A}R_{B})carbonyl, and (NR_{A}R_{B})sulfonyl.

The term “cycloalkylalkyl” as used herein, means a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “cycloalkyloxy” as used herein, means cycloalkyl group, as defined herein, appended to the parent molecular moiety through an oxygen atom, as defined herein. Representative examples of cycloalkyloxy include, but are not limited to, cyclopropyloxy, cyclobutyloxy, cyclopentyloxy, cyclohexyloxy, cycloheptyloxy, and cyclooctyloxy.

The term “cycloalkylthio” as used herein, means cycloalkyl group, as defined herein, appended to the parent molecular moiety through a sulfur atom, as defined herein. Representative examples of cycloalkylthio include, but are not limited to, cyclopropylthio, cyclobutylthio, cyclopentylthio, cyclohexylthio, cycloheptylthio, and cyclooctylthio.

The term “formyl” as used herein, means a —C(O)H group.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “haloalkylsulfonyl” as used herein, means a haloalkyl group, as defined herein, appended to the parent molecular moiety through a sulfonyl group. Representative examples of haloalkylsulfonyl include, but are not limited to, trifluoromethylsulfonyl and (pentafluoroethyl)sulfonyl.

The term “haloalkylthio” as used herein, means a haloalkyl group, as defined herein, appended to the parent molecular moiety through a sulfur atom. Representative examples of haloalkylthio include, but are not limited to, trifluoromethylthio and (pentafluoroethyl)thio.

The term “heteroaryl,” as used herein, refers to an aromatic five- or six-membered ring wherein 1, 2, 3, or 4 heteroatoms are independently selected from N, O, or S. The five membered rings have two double bonds and the six membered rings have three double bonds. The heteroaryl groups are connected to the parent molecular moiety through a carbon or nitrogen atom. Representative examples of heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetraazolyl, thiadiazolyl, thiazolyl, thienyl, triazolyl, and triazinyl.

The heteroaryl groups of the present invention are optionally substituted 1, 2, 3, or 4 substituents independently selected from alkenyl, alkoxy, alkoxyalkyl, alkoxycarbonyl, alkoxysulfonyl, alkyl, alkylcarbonyl, alkylcarbonyloxy, alkylsulfonyl, alkylthio, alkynyl, carboxy, cyano, formyl, haloalkoxy, haloalkyl, haloalkylthio, halogen, hydroxy, hydroxyalkyl, mercapto, nitro, —NR_{A}R_{B}, (NR_{A}R_{B})carbonyl, and (NR_{A}R_{B})sulfonyl.

The term “heteroarylalkyl” as used herein, means a heteroaryl, as defined herein, appended to the parent molecular moiety through an alkyl group, as defined herein. Representative examples of heteroarylalkyl include, but are not limited to, pyridin-3-ylmethyl and 2-pyrimidin-2-ylpropyl.

The term “heteroaryloxy” as used herein, means a heteroaryl group, as defined herein, appended to the parent molecular moiety through an oxygen atom. Representative examples of heteroaryloxy include, but are not limited to, pyridin-3-yloxy, pyridin-4-yloxy, and quinolin-3-yloxy.

The term “heteroarylthio” as used herein, means a heteroaryl group, as defined herein, appended to the parent molecular moiety through a sulfur atom. Representative examples of heteroarylthio include, but are not limited to, pyridin-3-ylthio, pyridin-4-ylthio, and quinolin-3-ylthio.

The term “heterocycle,” as used herein, refers to a three, four, five, six, seven or eight membered ring containing one or two heteroatoms independently selected from the group consisting of nitrogen, oxygen, and sulfur. The three membered ring has zero double bonds. The four and five membered ring has zero or one double bond. The six membered ring has zero, one, or two double bonds. The seven and eight membered rings have zero, one, two, or three double bonds. The heterocycle groups of the present invention can be attached to the parent molecular moiety through a carbon atom or a nitrogen atom. Representative examples of heterocycle include, but are not limited to, azetidinyl, azepanyl, aziridinyl, azocanyl, morpholinyl, piperazinyl, piperidinyl, pyrrolidinyl, and thiomorpholinyl.

The heterocycles of the present invention are optionally substituted withl, 2, 3, or 4 substituents independently selected from alkenyl, alkoxy, alkoxyalkyl, alkoxycarbonyl, alkoxysulfonyl, alkyl, alkylcarbonyl, alkylcarbonyloxy, alkylsulfonyl, alkylthio, alkynyl, carboxy, cyano, formyl, haloalkoxy, haloalkyl, haloalkylthio, halogen, hydroxy, hydroxyalkyl, mercapto, —NR_{A}R_{B}, (NR_{A}R_{B})carbonyl, and (NR_{A}R_{B})sulfonyl.

The term “heterocyclealkyl” as used herein, means a heterocycle, as defined herein, appended to the parent molecular moiety through an alkyl group, as defined herein. Representative examples of heterocyclealkyl include, but are not limited to, piperidin-1-ylmethyl and 2-piperidin-1-ylethyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “lower alkenyl” as used herein, is a subset of alkenyl, as defined herein, and means an alkenyl group containing from 2 to 4 carbon atoms. Examples of lower alkenyl are ethenyl, propenyl, and butenyl.

The term “lower alkoxy” as used herein, is a subset of alkoxy, as defined herein, and means a lower alkyl group, as defined herein, appended to the parent molecular moiety through an oxygen atom, as defined herein. Representative examples of lower alkoxy include, but are not limited to, methoxy, ethoxy, propoxy, 2-propoxy, butoxy, and tert-butoxy.

The term “lower alkyl” as used herein, is a subset of alkyl, as defined herein, and means a straight or branched chain hydrocarbon group containing from 1 to 4 carbon atoms. Representative examples of lower alkyl are methyl, ethyl, n-propyl, iso-propyl, n-butyl, iso-butyl, sec-butyl, and tert-butyl.

The term “lower alkylthio” as used herein, is a subset of alkylthio, and means a lower alkyl group, as defined herein, appended to the parent molecular moiety through a sulfur atom. Representative examples of lower alkylthio include, but are not limited, methylthio, ethylthio, and tert-butylthio.

The term “lower alkynyl” as used herein, is a subset of alkynyl, as defined herein, and means an alkynyl group containing from 2 to 4 carbon atoms. Examples of lower alkynyl are ethynyl, propynyl, and butynyl.

The term “lower haloalkoxy” as used herein, is a subset of haloalkoxy, as defined herein, and means a straight or branched chain haloalkoxy group containing from 1 to 4 carbon atoms. Representative examples of lower haloalkoxy include, but are not limited to, trifluoromethoxy, trichloromethoxy, dichloromethoxy, fluoromethoxy, and pentafluoroethoxy.

The term “lower haloalkyl” as used herein, is a subset of haloalkyl, as defined herein, and means a straight or branched chain haloalkyl group containing from 1 to 4 carbon atoms. Representative examples of lower haloalkyl include, but are not limited to, trifluoromethyl, trichloromethyl, dichloromethyl, fluoromethyl, and pentafluoroethyl.

The term “lower haloalkylthio” as used herein, is a subset of haloalkylthio, as defined herein, and means a straight or branched chain haloalkylthio group containing from 1 to 4 carbon atoms. Representative examples of lower haloalkylthio include, but are not limited to, trifluoromethylthio, trichloromethylthio, fluoromethylthio, and (pentafluoroethyl)thio.

The term “mercapto” as used herein, means a —SH group.

The term “nitro” as used herein, means a —NO₂ group.

The term “—NR_{A}R_{B}” as used herein, means two groups, R_{A} and R_{B}, which are appended to the parent molecular moiety through a nitrogen atom. R_{A} and R_{B} are each independently hydrogen or alkyl. Representative examples of —NR_{A}R_{B} include, but are not limited to, amino, methylamino, dimethylamino, and diethylamino.

The term “(NR_{A}R_{B})carbonyl” as used herein, means a —NR_{A}R_{B} group, as defined herein, appended to the parent molecular moiety through a carbonyl group, as defined herein. Representative examples of (NR_{A}R_{B})carbonyl include, but are not limited to, aminocarbonyl, (methylamino)carbonyl, (dimethylamino)carbonyl, and (ethylmethylamino)carbonyl.

The term “(NR_{A}R_{B})sulfonyl” as used herein, means a —NR_{A}R_{B} group, as defined herein, appended to the parent molecular moiety through a sulfonyl group, as defined herein. Representative examples of (NR_{A}R_{B})sulfonyl include, but are not limited to, aminosulfonyl, (methylamino)sulfonyl, (dimethylamino)sulfonyl, and (ethylmethylamino)sulfonyl.

The term “—NR_{C}R_{D}” as used herein, means two groups, R_{C} and R_{D}, which are appended to the parent molecular moiety through a nitrogen atom. R_{C} and R_{D} are each independently hydrogen, alkenyl, alkoxycarbonyl, alkyl, alkylcarbonyl, alkynyl, aryl, arylalkyl, cycloalkyl, cycloalkylalkyl, heteroaryl, heteroarylalkyl, or (NR_{A}R_{B})carbonyl. Representative examples of —NR_{C}R_{D} include, but are not limited to, amino, methylamino, dimethylamino, and ethylmethylamino.

The term “sulfonyl” as used herein, means a —S(O)₂— group.

Compounds of the present invention can exist as stereoisomers, wherein asymmetric or chiral centers are present. Stereoisomers are designated (R) or (S), depending on the configuration of substituents around the chiral carbon atom. The terms (R) and (S) used herein are configurations as defined in IUPAC 1974 Recommendations for Section E, Fundamental Stereochemistry, Pure Appl. Chem., (1976), 45: 13-30. The present invention contemplates various stereoisomers and mixtures thereof and are specifically included within the scope of this invention. Stereoisomers include enantiomers, diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution, a technique well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) direct separation of the mixture of optical enantiomers on chiral chromatographic columns, or (3) formation of a diastereomeric salt followed by selective recrystallization of one of the diastereomeric salts.

Compounds of the present invention were named by ACD/ChemSketch version 5.0 (developed by Advanced Chemistry Development, Inc., Toronto, ON, Canada) or were given names consistent with ACD nomenclature.

Preferred compounds of the present invention include:

N-(4-tert-butylphenyl)-3′-chloro-3,6-dihydro-2H-1,2′-bipyridine-4-carboxamide;

3′-chloro-N-(4-methylphenyl)-3,6-dihydro-2H-1,2′-bipyridine-4-carboxamide;

3′-chloro-N-(4-methoxyphenyl)-3,6-dihydro-2H-1,2′-bipyridine-4-carboxamide;

3′-chloro-N-(4-fluorophenyl)-3,6-dihydro-2H-1,2′-bipyridine-4-carboxamide;

3′-chloro-N-(4-chlorophenyl)-3,6-dihydro-2H-1,2′-bipyridine-4-carboxamide;

N-(4-bromophenyl)-3′-chloro-3,6-dihydro-2H-1,2′-bipyridine-4-carboxamide;

3′-chloro-N-[4-(trifluoromethoxy)phenyl]-3,6-dihydro-2H-1,2′-bipyridine-4-carboxamide;

3′-chloro-N-(4-phenoxyphenyl)-3,6-dihydro-2H-1,2′-bipyridine-4-carboxamide;

3′-chloro-N-(4-ethylphenyl)-3,6-dihydro-2H-1,2′-bipyridine-4-carboxamide;

3′-chloro-N-(4-isopropylphenyl)-3,6-dihydro-2H-1,2′-bipyridine-4-carboxamide;

N-(3-tert-butylphenyl)-3′-chloro-3,6-dihydro-2H-1,2′-bipyridine-4-carboxamide;

N-1,1′-biphenyl-4-yl-3′-chloro-3,6-dihydro-2H-1,2′-bipyridine-4-carboxamide;

3′-chloro-N-(4-propoxyphenyl)-3,6-dihydro-2H-1,2′-bipyridine-4-carboxamide;

3′-chloro-N-[4-(methylthio)phenyl]-3,6-dihydro-2H-1,2′-bipyridine-4-carboxamide;

3′-chloro-N-(3-fluoro-4-methylphenyl)-3,6-dihydro-2H-1,2′-bipyridine-4-carboxamide;

3′-chloro-N-[4-(trifluoromethyl)phenyl]-3,6-dihydro-2H-1,2′-bipyridine-4-carboxamide;

3′-chloro-N-(3-fluorophenyl)-3,6-dihydro-2H-1,2′-bipyridine-4-carboxamide;

3′-chloro-N-[4-(dimethylamino)phenyl]-3,6-dihydro-2H-1,2′-bipyridine-4-carboxamide;

3′-chloro-N-[4-(diethylamino)phenyl]-3,6-dihydro-2H-1,2′-bipyridine-4-carboxamide;

3′-chloro-N-[4-(1-piperidinyl)phenyl]-3,6-dihydro-2H-1,2′-bipyridine-4-carboxamide;

3′-chloro-N-[4-(4-morpholinyl)phenyl]-3,6-dihydro-2H-1,2′-bipyridine-4-carboxamide;

N-[4-(1-azepanyl)phenyl]-3′-chloro-3,6-dihydro-2H-1,2′-bipyridine-4-carboxamide;

N-(4-tert-butylphenyl)-3-(3-chloro-2-pyridinyl)-3,8-diazabicyclo[3.2.1]octane-8-carboxamide;

3-(3-chloro-2-pyridinyl)-N-(3,4-dichlorophenyl)-3,8-diazabicyclo[3.2.1]octane-8-carboxamide

3-(3-chloro-2-pyridinyl)-N-[3-(trifluoromethyl)phenyl]-3,8-diazabicyclo[3.2.1]octane-8-carboxamide;

3-(3-chloro-2-pyridinyl)-N-[4-(trifluoromethyl)phenyl]-3,8-diazabicyclo[3.2.1]octane-8-carboxamide;

3-(3-chloro-2-pyridinyl)-N-(4-fluorophenyl)-3,8-diazabicyclo[3.2.1]octane-8-carboxamide;

N-(4-chlorophenyl)-3-(3-chloro-2-pyridinyl)-3,8-diazabicyclo[3.2.1]octane-8-carboxamide;

N-(4-bromophenyl)-3-(3-chloro-2-pyridinyl)-3,8-diazabicyclo[3.2.1]octane-8-carboxamide;

3-(3-chloro-2-pyridinyl)-N-(4-iodophenyl)-3,8-diazabicyclo[3.2.1]octane-8-carboxamide;

N-(4-butylphenyl)-3-(3-chloro-2-pyridinyl)-3,8-diazabicyclo[3.2.1]octane-8-carboxamide;

3-(3-chloro-2-pyridinyl)-N-(4-isopropylphenyl)-3,8-diazabicyclo[3.2.1]octane-8-carboxamide;

3-(3-chloro-2-pyridinyl)-N-{4-[(trifluoromethyl)thio]phenyl}-3,8-diazabicyclo[3.2.1]octane-8-carboxamide;

N-(4-tert-butylphenyl)-8-(3-chloro-2-pyridinyl)-3,8-diazabicyclo[3.2.1]octane-3-carboxamide;

8-(3-chloro-2-pyridinyl)-N-[4-(trifluoromethyl)phenyl]-3,8-diazabicyclo[3.2.1]octane-3-carboxamide;

N-(4-tert-butylphenyl)-1-(3-chloro-2-pyridinyl)-(cis)-3-hydroxy-4-piperidinecarboxamide;

N-(4-tert-butylphenyl)-1-(3-chloro-2-pyridinyl)-(trans)-3-hydroxy-4-piperidinecarboxamide;

1-(3-chloro-2-pyridinyl)-4-hydroxy-N-[4-(trifluoromethyl)phenyl]-4-piperidinecarboxamide;

N-(4-tert-butylphenyl)-3′-(trifluoromethyl)-3,6-dihydro-2H-1,2′-bipyridine-4-carboxamide;

N-(4-chlorophenyl)-3′-(trifluoromethyl)-3,6-dihydro-2H-1,2′-bipyridine-4-carboxamide;

N-[4-(trifluoromethoxy)phenyl]-3′-(trifluoromethyl)-3,6-dihydro-2H-1,2′-bipyridine-4-carboxamide;

3′-(trifluoromethyl)-N-{4-[(trifluoromethyl)thio]phenyl}-3,6-dihydro-2H-1,2′-bipyridine-4-carboxamide;

3′-(trifluoromethyl)-N-{4-[(trifluoromethyl)sulfonyl]phenyl}-3,6-dihydro-2H-1,2′-bipyridine-4-carboxamide;

N-(3-fluoro-4-methylphenyl)-3′-(trifluoromethyl)-3,6-dihydro-2H-1,2′-bipyridine-4-carboxamide;

N-(4-chlorophenyl)-1-pyrimidin-2-yl-1,2,3 ,6-tetrahydropyridine-4-carboxamide;

1-pyrimidin-2-yl-N-{4-[(trifluoromethyl)thio]phenyl}-1,2,3,6-tetrahydropyridine-4-carboxamide; and

1-pyrimidin-2-yl-N-{4-[(trifluoromethyl)sulfonyl]phenyl}-1,2,3,6-tetrahydropyridine-4-carboxamide; or pharmaceutically acceptable salts, esters, amides, or prodrugs thereof.

Abbreviations

Abbreviations which have been used in the descriptions of the Schemes and the Examples that follow are: Ac for acetyl; Bu for butyl; DCC for 1,3-dicyclohexylcarbodiimide; DIEA for diisopropylethylamine; DMAP for 4-dimethylaminopyridine; DME for 1,2-dimethoxyethane; DMF for N,N-dimethylformamide; DMSO for dimethylsulfoxide; EDCI or EDC for 1-ethyl-3-[3-(dimethylamino)propyl]-carbodiimide hydrochloride; Ph for phenyl; TFA for trifluoroacetic acid; THF for tetrahydrofuran; and Tf for —S(O)₂CF₃.

Preparation of Compounds of the Present Invention

The compounds and processes of the present invention will be better understood in connection with the following synthetic Schemes and Examples which illustrate a means by which the compounds of the present invention can be prepared. Further, all citations herein are incorporated by reference.

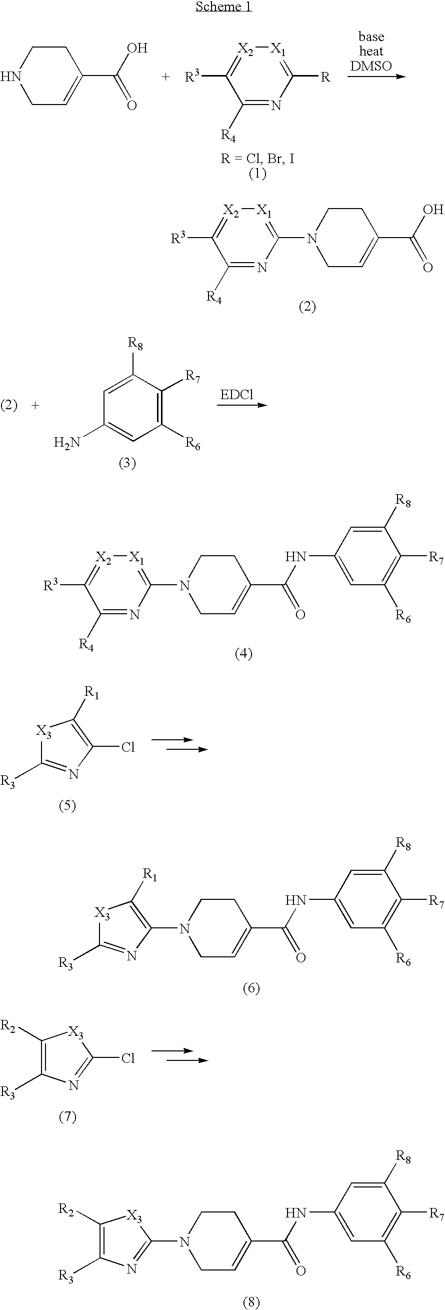

Amides of general formula (4), wherein X₁, X₂, X₃, R₃, R₄, R₆, R₇, and R₈ are as defined in formula (I-IV), can be prepared as described in Scheme 1. 1,2,3,6-Tetrahydropyridine-4-carboxylic acid, prepared as described in Examples 1A-1C herein, can be treated with 2-halo compounds of general formula (1) and a base including, but not limited to potassium carbonate, in a solvent including, but not limited to, DMSO or DMF and heated until reaction is complete to provide acids of general formula (2). Acids of general formula (2) can be coupled to anilines of general formula (3) using EDCI or DCC to provide amides of general formula (4).

Amides of general formula (6) and general formula (8), wherein X₃, R₁, R₂, R₃, R₆, R₇, and R₈ are as defined in formula (I-IV), can be prepared as described in Scheme 1. Oxazoles or thiazoles of general formula (5) or general formula (7) can be processed in a similar manner as compounds of general formula (1) in Scheme 1 to provide amides of general formula (6) or amides of general formula (8).

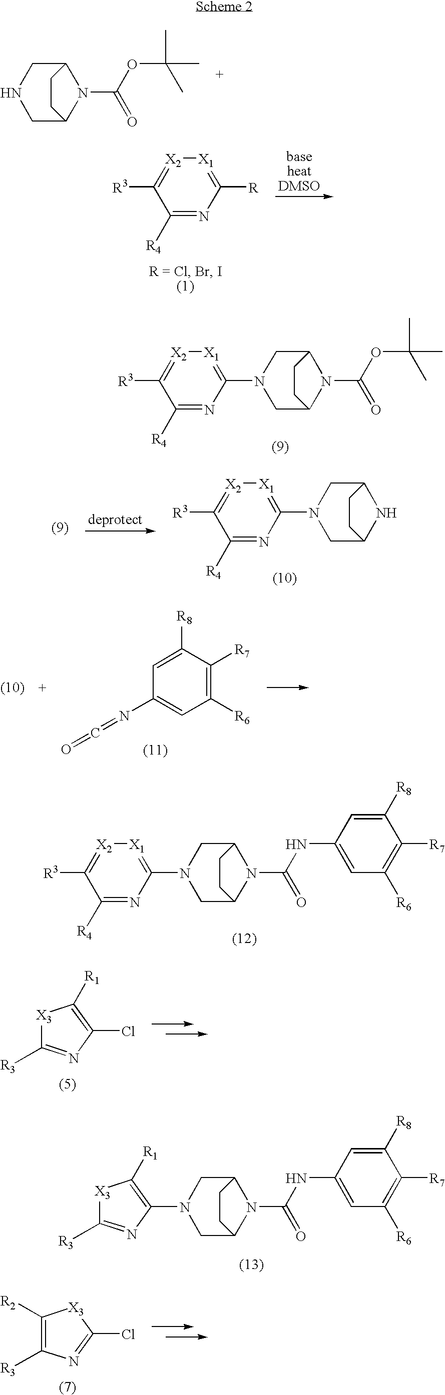

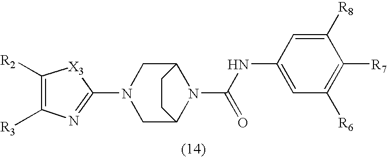

Amides of general formula (12), wherein X₁, X₂, X₃, R₃, R₄, R₆, R₇, and R₈ are as defined in formula (I-IV), can be prepared as described in Scheme 2. tert-Butyl 3,8-diazabicyclo[3.2.1]octane-8-carboxylate, prepared as described in Examples 23A-23I herein, can be treated with 2-halo compounds of general formula (1) as described in Scheme 1 to provide compounds of general formula (9). Compounds of general formula (9) can be deprotected with trifluoroacetic acid in methylene chloride (1:1) or with 4.5N hydrochloric acid in 1,4-dioxane to provide compounds of general formula (10). Compounds of general formula (10) can be treated with isocyanates of general formula (11) to provide amides of general formula (12).

Amides of general formula (13) and amides of general formula (14), wherein X₃, R₁, R₂, R₃, R₆, R₇, and R₈ are as defined in formula (I-V), can be prepared as described in Scheme 2. Oxazoles or thiazoles of general formula (5) or general formula (7) can be processed in a similar manner as compounds of general formula (1) in Scheme 2 to provide amides of general formula (13) or amides of general formula (14).

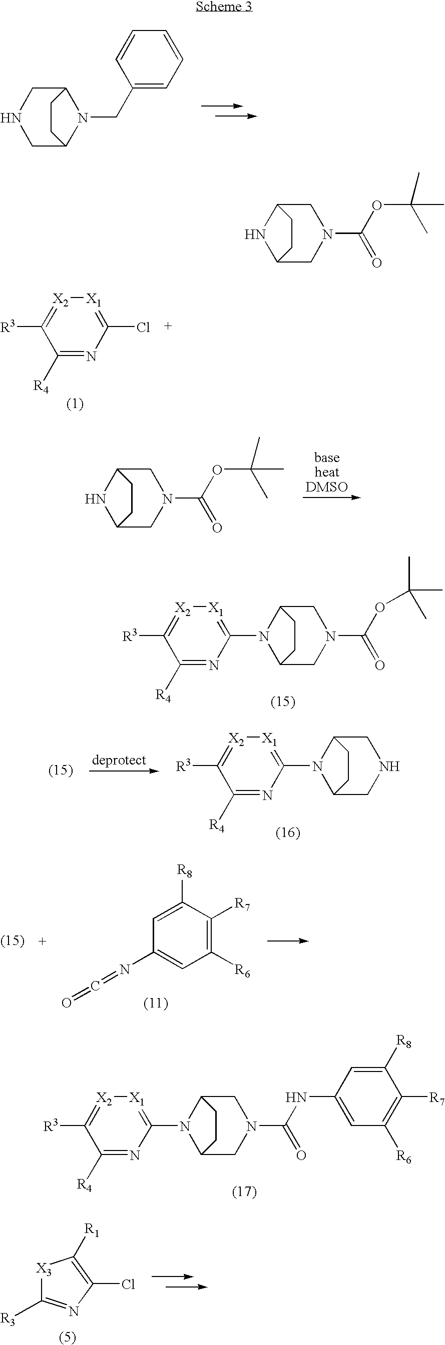

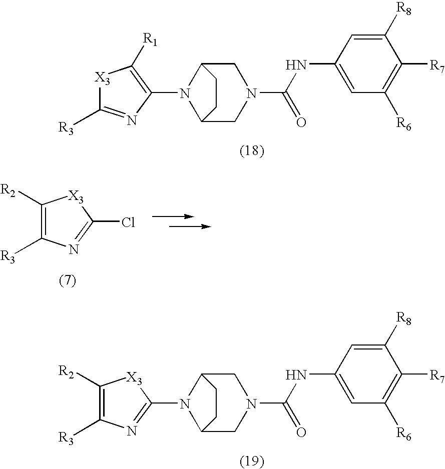

Amides of general formula (17), wherein X₁, X₂, X₃, R₃, R₄, R₆, R₇, and R₈ are as defined in formula (I-IV), can be prepared as described in Scheme 3. 8-Benzyl-3,8-diazabicyclo[3.2.1]octane, prepared as described in Examples 23A-23F herein, can be processed as described in Examples 34A-34C to provide tert-butyl 3,8-diazabicyclo[3.2.1]octane-3-carboxylate. tert-Butyl 3,8-diazabicyclo[3.2.1]octane-3-carboxylate can be treated with 2-halo compounds of general formula (1) as described in Scheme 1 to provide compounds of general formula (15). Compounds of general formula (15) can be deprotected with trifluoroacetic acid in methylene chloride (1:1) or with 4.5N hydrochloric acid in 1,4-dioxane to provide compounds of general formula (16). Compounds of general formula (16) can be treated with isocyanates of general formula (11) to provide amides of general formula (17).

Amides of general formula (18) and amides of general formula (19), wherein X₃, R₁, R₂, R₃, R₆, R₇, and R₈ are as defined in formula (I-IV), can be prepared as described in Scheme 3. Oxazoles or thiazoles of general formula (5) or general formula (7) can be processed in a similar manner as compounds of general formula (1) in Scheme 3 to provide amides of general formula (18) or amides of general formula (19).

### EXAMPLE 1

### N-(4-tert-butylphenyl)-3′-chloro-3,6-dihydro-2H-1,2′-bipyridine-4-carboxamide

### Example 1A

### 1-(1-chloroethyl) 4-ethyl 3,6-dihydro-1,4(2H)-pyridinedicarboxylate

Ethyl 1-methyl-1,2,3,6-tetrahydro-4-pyridinecarboxylate (47 g, 278 mmol) in dichloroethane (800 mL) was treated with a-chloroethyl chloroformate (50 g, 350 mmol) dropwise at 0° C. The mixture was warmed to room temperature, refluxed for 2 hours, and then allowed to cool to room temperature. The mixture was concentrated under reduced pressure and the residue was purified via column chromatography (SiO₂, ethyl acetate) to provide the title compound. ¹H NMR (300 MHz, CDCl₃) δ 6.88 (m, 1H), 6.61 (q, 1H), 4.22 (q, 2H), 4.16 (m, 2H), 7.35 (d, 2H), 3.58 (m, 2H), 2.44 (m, 2H), 1.82 (d, 3H)

### EXAMPLE 1B

### ethyl 1,2,3,6-tetrahydro-4-pyridinecarboxylate

1-(1-Chloroethyl) 4-ethyl 3,6-dihydro-1,4(2H)-pyridinedicarboxylate (61.8 g, 236 mmol) in methanol (500 mL) was heated at reflux for 30 minutes. The mixture was allowed to cool to room temperature and concentrated to provide the title compound. ¹H NMR (300 MHz, CDCl₃) δ 6.86 (m, 1H), 4.22 (q, 2H), 3.85 (m, 2H), 3.34 (m, 2H), 2.75 (m, 2H), 1.30 (t, 3H).

### EXAMPLE 1C

### 1,2,3,6-tetrahydropyridine-4-carboxylic acid

Ethyl 1,2,3,6-tetrahydro-4-pyridinecarboxylate (44.7 g, 234 mmol) in dichloromethane (200 mL) was treated with aqueous K₂CO₃ (saturated) and the phases were separated. The aqueous layer was dried under reduced pressure and the residue was triturated with dichloromethane (100×3 mL) and dissolved in methanol (100 mL). The solution was treated with a saturated solution of NaOH (10 g, 250 mmol) in methanol (250 mL) and sodium methoxide in methanol (0.5M, 200 mL, 100 mmol). After stirring at room temperatrue for two days, the mixture was concentrated under reduced pressure. The residue was triturated with methanol (200×5 mL) and dried under reduced pressure to provide the title compound. ¹H NMR (300 MHz, CD₃OD) δ 6.60 (m, 1H), 3.38 (m, 2H), 2.88 (m, 2H), 2.30 (m, 2H).

### EXAMPLE 1D

### 3′-chloro-3,6-dihydro-2H-1,2′-bipyridine-4-carboxylic acid

1,2,3,6-Tetrahydro-4-pyridinecarboxylic acid (7.0 g, 47 mmol) and 2-bromo-3-chloropyridine (11.0 g, 57 mmol) were combined in DMSO (100 mL) and treated with anhydrous K₂CO₃ (8.0 g, 58 mmol). The mixture was heated at 90° C. overnight, allowed to cool to room temperature, and then concentrated under reduced pressure. The residue in dichloromethane (200 mL) was cooled to −78° C. and treated with an excess of TFA (10 mL, 130 mmol) dropwise. The mixture was allowed to warm to room temperature and the layers were separated. The organic layer was washed with brine, dried over Na₂SO₄, filtered, and the filtrate concentrated under reduced pressure to provide the title compound which was used in the next step without further purification.

### EXAMPLE 1E

### N-(4-tert-butylphenyl)-3′-chloro-3,6-dihydro-2H-1,2′-bipyridine-4-carboxamide

3′-Chloro-3,6-dihydro-2H-1,2′-bipyridine-4-carboxylic acid (120 mg, 0.35 mmol), 4-tert-butylaniline (90.0 mg, 0.6 mmol), and EDCI (145 mg, 0.75 mmol) were combined in dichloromethane (3 mL) under N₂ and stirred at room temperature overnight. The mixture was treated with water and the layers were separated. The organic layer was concentrated and the residue was purified via column chromatography (SiO₂, ethyl acetate:hexanes, 1:4) to provide the title compound. ¹H NMR (500 MHz, CDCl₃) δ 8.18 (dd, 1H), 7.61 (dd, 1H), 7.46 (d, 2H), 7.38 (s(br), 1H), 7.35 (d, 2H), 6.85 (dd, 1H), 6.75 (m, 1H), 4.10 (dd, 2H), 3.58 (dd, 2H), 2.68 (m, 2H), 1.30 (s, 9H); MS (ESI) 370 (M+H)⁺.

### EXAMPLE 2

### 3′-chloro-N-(4-methylphenyl)-3,6-dihydro-2H-1,2′-bipyridine-4-carboxamide

The title compound was prepared using the procedure described in Example 1E using 4-methylaniline instead of 4-tert-butylaniline. ¹H NMR (500 MHz, CDCl₃) δ 8.18 (dd, 1H), 7.62 (dd, 1H), 7.43 (d, 2H), 7.10 (1H, NH), 7.15 (d, 2H), 6.84 (dd, 1H), 6.76 (m, 1H), 4.08 (dd, 2H), 3.58 (dd, 2H), 2.66 (m, 2H), 2.32 (s, 3H); MS (ESI) 328 (M+H)⁺.

### EXAMPLE 3

### 3′-chloro-N-(4-methoxyphenyl)-3,6-dihydro-2H-1,2′-bipyridine-4-carboxamide

The title compound was prepared using the procedure described in Example 1E using 4-methoxyaniline instead of 4-tert-butylaniline. ¹H NMR (500 MHz, CDCl₃) δ 8.21 (dd, 1H), 7.64 (dd, 1H), 7.55 (s(br) 1H), 7.45 (m, 2H), 6.90 (m, 3H), 6.76 (m, 1H), 4.15 (dd, 2H), 3.80 (s, 3H), 3.65 (dd, 2H), 2.68 (m, 2H); MS (ESI) 343 (M+H)⁺.

### EXAMPLE 4

### 3′-chloro-N-(4-fluorophenyl)-3,6-dihydro-2H-1,2′-bipyridine-4-carboxamide

The title compound was prepared using the procedure described in Example 1E using 4-fluoroaniline instead of 4-tert-butylaniline. ¹H NMR (500 MHz, CDCl₃) δ 8.20 (dd, 1H), 7.66 (dd, 1H), 7.58 (s(br) 1H), 7.54 (m, 2H), 7.02 (m, 2H), 6.90 (dd, 1H), 6.76 (m, 1H), 4.15 (dd, 2H), 3.62 (dd, 2H), 2.68 (m, 2H); MS (ESI) 331 (M+H)⁺.

### EXAMPLE 5

### 3′-chloro-N-(4-chlorophenyl)-3,6-dihydro-2H-1,2′-bipyridine-4-carboxamide

4-Chloroaniline (13.2 g, 103 mmol), 3′-chloro-3,6-dihydro-2H-1,2′-bipyridine-4-carboxylic acid (16.49 g, 69.1 mmol), 1-[3-(dimethylamino) propyl]-3-ethylcarbodiimide hydrochloride (26.5 g, 138 mmol) were combined in dichloromethane (150 mL) and stirred overnight at ambient temperature. The mixture was diluted with dichloromethane (200 mL), washed with water (400 mL), 400 mL brine, dried with sodium sulfate, filtered, and the filtrate was concentrated under reduced pressure. The residue was purified by chromatography on silica gel using hexane:ethyl acetate (7:3) to provide the title compound. ¹H NMR (500 MHz, CDCl₃) δ 8.20 (dd, 1H), 7.70 (dd, 1H), 7.62 (s(br) 1H), 7.54 (d, 2H), 7.30 (d, 2H), 6.92 (dd, 1H), 6.76 (m, 1H), 4.16 (dd, 2H), 3.64 (dd, 2H), 2.66 (m, 2H); MS (ESI) 349 (M+H)⁺.

### EXAMPLE 6

### N-(4-bromophenyl)-3′-chloro-3,6-dihydro-2H-1,2′-bipyridine-4-carboxamide

The title compound was prepared using the procedure described in Example 1E using 4-bromoaniline instead of 4-tert-butylaniline. ¹H NMR (500 MHz, CDCl₃) δ 8.20 (dd, 1H), 7.65 (dd, 1H), 7.55 (s(br) 1H), 7.48 (d, 2H), 7.44 (d, 2H), 6.88 (dd, 1H), 6.78 (m, 1H), 4.15 (dd, 2H), 3.61 (dd, 2H), 2.66 (m, 2H); MS (ESI) 393 (M+H)⁺.

### EXAMPLE 7

### 3′-chloro-N-[4-(trifluoromethoxy)phenyl]-3,6-dihydro-2H-1,2′-bipyridine-4-carboxamide

The title compound was prepared using the procedure described in Example 1E using 4-(trifluoromethoxy)aniline instead of 4-tert-butylaniline. ¹H NMR (500 MHz, CDCl₃) δ 8.20 (dd, 1H), 7.65 (dd, 1H), 7.55 (s(br) 1H), 7.48 (d, 2H), 7.44 (d, 2H), 6.88 (dd, 1H), 6.78 (m, 1H), 4.15 (dd, 2H), 3.61 (dd, 2H), 2.66 (m, 2H); MS (ESI) 393 (M+H)⁺.

### EXAMPLE 8

### 3′-chloro-N-(4-phenoxyphenyl)-3,6-dihydro-2H-1,2′-bipyridine-4-carboxamide

The title compound was prepared using the procedure described in Example 1E using 4-phenoxyaniline instead of 4-tert-butylaniline. ¹H NMR (500 MHz, CDCl₃) δ 8.19 (dd, 1H), 7.62 (dd, 1H), 7.54 (d, 2H), 7.54 (s(br) 1H), 7.32 (dd. 2H), 7.08 (t, 1H), 7.00 (m, 4H), 6.85 (dd, 1H), 6.78 (m, 1H), 4.12 (dd, 2H), 3.59 (dd, 2H), 2.68 (m, 2H); MS (ESI) 406 (M+H)⁺.

### EXAMPLE 9

### 3′-chloro-N-(4-ethylphenyl)-3,6-dihydro-2H-1,2′-bipyridine-4-carboxamide

The title compound was prepared using the procedure described in Example 1E using 4-ethylaniline instead of 4-tert-butylaniline. ¹H NMR (500 MHz, CDCl₃) δ 8.19 (dd, 1H), 7.62 (dd, 1H), 7.45 (d, 2H), 7.43 (s(br) 1H), 7.18 (dd. 2H), 6.84 (dd, 1H), 6.75 (m, 1H), 4.12 (dd, 2H), 3.78 (dd, 2H), 2.69 (m, 2H), 2.62 (q, 2H), 1.22 (t, 3H); MS (ESI) 342 (M+H)⁺.

### EXAMPLE 10

### 3′-chloro-N-(4-isopropylphenyl)-3,6-dihydro-2H-1,2′-bipyridine-4-carboxamide

The title compound was prepared using the procedure described in Example 1E using 4-isopropylaniline instead of 4-tert-butylaniline. ¹H NMR (500 MHz, CDCl₃) δ 8.19 (dd, 1H), 7.61 (dd, 1H), 7.50 (s(br), 1H), 7.46 (d, 2H), 7.18 (dd. 2H), 6.84 (dd, 1H), 6.76 (m,1H), 4.08 (dd, 2H), 3.56 (dd, 2H), 2.85 (m, 1H), 2.68 (m, 2H), 1.22 (d, 6H); MS (ESI) 356 (M+H)⁺.

### EXAMPLE 11

### N-(3-tert-butylphenyl)-3′-chloro-3,6-dihydro-2H-1,2′-bipyridine-4-carboxamide

The title compound was prepared using the procedure described in Example 1E using 3-tert-butylaniline instead of 4-tert-butylaniline. ¹H NMR (500 MHz, CDCl₃) δ 8.19 (dd, 1H), 7.64 (dd, 1H), 7.54 (m, 1H), 7.49 (s(br), 1H), 7.43 (m, 1H), 7.28 (d, 1H), 7.16 (m, 1H), 6.86 (dd, 1H), 6.77 (m, 1H), 4.12 (dd, 2H), 3.60 (dd, 2H), 2.70 (m, 2H), 1.32 (s, 9H); MS (ESI) 369 (M+H)⁺.

### EXAMPLE 12

### N-1,1′-biphenyl-4-yl-3′-chloro-3,6-dihydro-2H-1,2′-bipyridine-4-carboxamide

The title compound was prepared using the procedure described in Example 1E using 1,1′-biphenyl-4-ylamine instead of 4-tert-butylaniline. ¹H NMR (500 MHz, CDCl₃) δ 8.19 (dd, 1H), 7.62 (m, 3H), 7.58 (m, 5H), 7.42 (dd, 2H), 7.32 (t, 1H), 6.86 (dd, 1H), 6.77 (m, 1H), 4.12 (dd, 2H), 3.60 (dd, 2H), 2.70 (m, 2H); MS (ESI) 390 (M+H)⁺.

### EXAMPLE 13

### 3′-chloro-N-(4-propoxyphenyl)-3,6-dihydro-2H-1,2′-bipyridine-4-carboxamide

The title compound was prepared using the procedure described in Example 1E using 4-propoxyaniline instead of 4-tert-butylaniline. ¹H NMR (500 MHz, CDCl₃) δ 8.19 (dd, 1H), 7.60 (dd, 1H), 7.42 (m, 2H), 7.40 (s(br), 1H), 6.85 (m, 3H), 6.75 (m, 1H), 4.08 (dd, 2H), 3.90 (m, 2H), 3.58 (dd, 2H), 2.65 (m, 2H), 1.80 (m, 2H), 1.02 (m, 3H); MS (ESI) 372 (M+H)⁺.

### EXAMPLE 14

### 3′-chloro-N-[4-(methylthio)phenyl]-3,6-dihydro-2H-1,2′-bipyridine-4-carboxamide

The title compound was prepared using the procedure described in Example 1E using 4-(methylthio)aniline instead of 4-tert-butylaniline. ¹H NMR (500 MHz, CDCl₃) δ 8.20 (dd, 1H), 7.64 (dd, 1H), 7.52 (m, 3H), 7.25 (d, 2H), 6.86 (dd, 1H), 6.76 (m, 1H), 4.12 (dd, 2H), 3.60 (dd, 2H), 2.68 (m, 2H), 2.46 (s, 3H); MS (ESI) 360 (M+H)⁺.

### EXAMPLE 15

### 3′-chloro-N-(3-fluoro-4-methylphenyl)-3,6-dihydro-2H-1,2′-bipyridine-4-carboxamide

The title compound was prepared using the procedure described in Example 1E using 3-fluoro-4-methylaniline instead of 4-tert-butylaniline. ¹H NMR (500 MHz, CDCl₃) δ 8.19 (dd, 1H), 7.62 (dd, 1H), 7.48 (m, 2H), 7.10 (m, 2H), 6.85 (dd, 1H), 6.77 (m, 1H), 4.12 (dd, 2H), 3.58 (dd, 2H), 2.65 (m, 2H), 2.22 (s, 3H); MS (ESI) 346 (M+H)⁺.

### EXAMPLE 16

### 3′-chloro-N-[4-(trifluoromethyl)phenyl]-3,6-dihydro-2H-1,2′-bipyridine-4-carboxamide

The title compound was prepared using the procedure described in Example 1E using 4-(trifluoromethyl)aniline instead of 4-tert-butylaniline. ¹H NMR (500 MHz, CDCl₃) δ 8.19 (dd, 1H), 7.72 (d, 2H), 7.64 (dd, 1H), 7.60 (d, 2H), 7.58 (s(br), 1H), 6.86 (dd, 1H), 6.82 (m, 1H), 4.14 (dd, 2H), 3.59 (dd, 2H), 2.68 (m, 2H); MS (ESI) 382 (M+H)⁺.

### EXAMPLE 17

### 3′-chloro-N-(3-fluorophenyl)-3,6-dihydro-2H-1,2′-bipyridine-4-carboxamide

The title compound was prepared using the procedure described in Example 1E using 3-fluoroaniline instead of 4-tert-butylaniline. ¹H NMR (500 MHz, CDCl₃) δ 8.19 (dd, 1H), 7.65 (d, 2H), 7.59 (s(br), 1H), 7.54 (m, 1H), 7.24 (m, 1H), 7.19 (m, 1H), 6.86 (dd, 1H), 6.82 (m, 1H), 6.78 (m, 1H), 4.14 (dd, 2H), 3.60 (dd, 2H), 2.66 (m, 2H); MS (ESI) 332 (M+H)⁺.

### EXAMPLE 18

### 3′-chloro-N-[4-(dimethylamino)phenyl]-3,6-dihydro-2H-1,2′-bipyridine-4-carboxamide

The title compound was prepared using the procedure described in Example 1E using N,N-dimethyl-1,4-benzenediamine instead of 4-tert-butylaniline. ¹H NMR (500 MHz, CDCl₃) δ 8.24 (br s, 1H), 8.20 (dd, 1H), 7.76 (m, 3H), 7.48 (d, 2H), 6.85 (dd, 1H), 6.82 (m, 1H), 4.19 (dd, 2H), 3.68 (dd, 2H), 3.17 (s, 6H), 2.68 (m, 2H); MS (ESI) 357 (M+H)⁺.

### EXAMPLE 19

### 3′-chloro-N-[4-(diethylamino)phenyl]-3,6-dihydro-2H-1,2′-bipyridine-4-carboxamide

The title compound was prepared using the procedure described in Example 1E using N,N-diethyl-1,4-benzenediamine instead of 4-tert-butylaniline. ¹H NMR (500 MHz, CDCl₃) δ 8.21 (dd, 1H), 8.04 (s(br), 1H), 7.81 (d, 2H), 7.74 (dd, 1H), 7.51 (d, 2H), 6.92 (dd, 1H), 6.82 (m, 1H), 4.20 (dd, 2H), 3.68 (dd, 2H), 3.52 (m, 4H), 2.69 (m, 2H), 1.15 (t, 6H); MS (ESI) 385 (M+H)⁺.

### EXAMPLE 20

### 3′-chloro-N-[4-(1-piperidinyl)phenyl]-3,6-dihydro-2H-1,2′-bipyridine-4-carboxamide

The title compound was prepared using the procedure described in Example 1E using 4-(1-piperidinyl)aniline instead of 4-tert-butylaniline. ¹H NMR (500 MHz, CDCl₃) δ 8.41 (br s, 1H), 8.20 (dd, 1H), 7.75 (d, 2H), 7.72 (dd, 1H), 7.50 (d, 2H), 6.92 (dd, 1H), 6.82 (m,1H), 4.16 (dd, 2H), 3.64 (dd, 2H), 3.44 (m, 4H), 2.69 (m, 2H), 2.08 (m, 4H), 1.72 (m, 2H); MS (ESI) 397 (M+H)⁺.

### EXAMPLE 21

### 3′-chloro-N-[4-(4-morpholinyl)phenyl]-3,6-dihydro-2H-1,2′-bipyridine-4-carboxamide

The title compound was prepared using the procedure described in Example 1E using 4-(4-morpholinyl)aniline instead of 4-tert-butylaniline. ¹H NMR (500 MHz, CDCl₃) δ 8.42 (s(br), 1H), 8.20 (dd, 1H), 7.92 (dd, 1H), 7.76 (d, 2H), 7.44 (d, 2H), 7.04 (dd, 1H), 6.76 (m, 1H), 4.28 (m, 2H), 4.10 (m, 4H), 3.81 (dd, 2H), 3.52 (m, 4H), 2.71 (m, 2H); MS (ESI) 399 (M+H)⁺.

### EXAMPLE 22

### N-[4-(1-azepanyl)phenyl]-3′-chloro-3,6-dihydro-2H-1,2′-bipyridine-4-carboxamide

The title compound was prepared using the procedure described in Example 1E using 4-(1-azepanyl)aniline instead of 4-tert-butylaniline. ¹H NMR (500 MHz, CDCl₃) δ 8.58 (s(br), 1H), 8.20 (dd, 1H), 7.86 (dd, 1H), 7.74 (d, 2H), 7.45 (d, 2H), 7.01 (dd, 1H), 6.78 (m, 1H), 4.24 (m, 2H), 3.75 (dd, 2H), 3.62 (m, 4H), 2.70 (m, 2H), 2.08 (m, 4H), 1.82 (m, 4H); MS (ESI) 411 (M+H)⁺.

### EXAMPLE 23

### N-(4-tert-butylphenyi)-3-(3-chloro-2-pyridinyl)-3,8-diazabicyclo[3.2.1]octane-8-carboxamide

### EXAMPLE 23A

### methyl 1-benzyl-5-oxo-2-pyrrolidinecarboxylate

1-Benzyl-5-oxo-2-pyrrolidinecarboxylic acid (9.69 g, 44.2 mmol) in methanol (75 mL) was treated with sulfuric acid (0.5 mL) and refluxed for 2.5 hours. The mixture was allowed to cool to room temperature and concentrated under reduced pressure. The residue was dissolved in ethyl acetate and washed with saturated sodium bicarbonate solution. The ethyl acetate phase was concentrated under reduced pressure to provide the title compound which was used in the next step without further purification. MS (ESI) m/z: 234 (M+H)⁺; ¹H NMR (CDCl₃) δ 2.08 (m, 1H), 2.25 (m, 1H), 2.43 (m, 1H), 2.55 (m, 1H), 3.68 (s, 3H), 3.99 (dd, 1H), 4.01 (d, 1H), 5.02 (d, 1H), 7.22 (m, 2H), 7.30 (m, 3H).

### EXAMPLE 23B

### methyl 1-benzyl-5-thioxo-2-pyrrolidinecarboxylate

Methyl 1-benzyl-5-oxo-2-pyrrolidinecarboxylate (18.15 g, 77.8mmol) and Lawesson's reagent (31.5 g, 77.9mmol) were combined in dry tetrahydrofuran (100 mL) and stirred overnight at room temperature. The mixture was filtered, the filter cake was rinsed with tetrahydrofuran, and the filtrate was concentrated under reduced pressure. The residue was dissolved in ethyl acetate and washed with saturated sodium bicarbonate solution. The organic phase was concentrated under reduced pressure and the residue was purified by chromatography on silica gel (3:1, hexanes:ethyl acetate) to provide the title compound. MS (ESI) m/z: 250 (M+H)⁺; ¹H NMR (CDCl₃) δ 2.17 (m, 1H), 2.27 (m, 1H), 3.15 (m, 2H), 3.69 (s, 3H), 4.30 (dd, 1H), 4.37 (d, 1H), 5.73 (d, 1H), 7.32 (m, 5H).

### EXAMPLE 23C

### 1-benzyl-2-(methoxycarbonyl)-5-(methylthio)-3,4-dihydro-2H-pyrrolium iodide

Methyl 1-benzyl-5-thioxo-2-pyrrolidinecarboxylate (16.5 g, 66.2 mmol) in iodomethane (70 mL) was stirred overnight at room temperature. The mixture was concentrated under reduced pressure to provide the title compound which was used in the next step without further purification. ¹H NMR (CDCl₃) δ 2.24 (m, 1H), 3.04 (s, 3H), 3.16 (m, 1H), 3.25 (m, 1H), 3.63 (s, 3H), 4.29 (dd, 1H), 4.72 (d, 1H), 4.89 (dd, 1H), 5.14 (d, 1H), 7.43 (m, 3H), 7.51 (m, 2H).

### EXAMPLE 23D

### methyl 1-benzyl-5-(nitromethylene)-2-pyrrolidinecarboxylate

1-Benzyl-2-(methoxycarbonyl)-5-(methylthio)-3,4-dihydro-2H-pyrrolium iodide (25.68 g, 65.6 mmol) in dry N,N-dimethylformamide (80 mL) was treated with nitromethane (17.8 mL, 328 mmol) and diisopropylethyl amine (12.6 mL, 72.2 mmol) and stirred overnight at room temperature. The mixture was heated at 60° C. for 5 hours, allowed to cool to room temperature, and concentrated under reduce pressure. The residue was purified by chromatography on silica gel (3:2 hexanes:ethyl acetate) to provide the title compound. MS (ESI) m/z: 277 (M+H)⁺; ¹H NMR (CDCl₃) δ 2.22 (m, 1H), 2.34 (m, 1H), 3.39 (5, 1H), 3.69 & 3.75 (dd & dd, 1H), 3.72 (s, 3H), 4.24 (dd, 1H), 4.30 (d, 1H), 4.51 (d, 1H), 6.87 (s, 1H), 7.16 (dd, 2H), 7.35 (m, 3H).

### EXAMPLE 23E

### 8-benzyl-3,8-diazabicyclo[3.2.1]octane-2-one

Methyl 1-benzyl-5-(nitromethylene)-2-pyrrolidinecarboxylate (9.92 g, 35.9 mmol) and 5% platinum on activated carbon (11.25 g) were combined in methanol (100 mL) and shaken for 56 hours at ambient temperature under a hydrogen atmosphere (60 psi). The misture was filtered and the filtrate concentrated to provide the title compound. MS (DCI/NH₃) m/z: 217 (M+H)⁺.

### EXAMPLE 23F

### 8-benzyl-3,8-diazabicyclo[3.2.1]octane

8-Benzyl-3,8-diazabicyclo[3.2.1]octan-2-one (5.87 g, 27.1 mmol) in dry tetrahydroftiran (40 mL) was treated with 1M lithium aluminum hydride in THF (81.4 mL), heated at 60° C. for 2 hours, and then allowed to cool to room temperature and stir overnight. The mixture was cooled to 0° C. and treated in succession with water (4.4 mL), tetrahydrofuran (200 mL), 15% sodium hydroxide solution (4.4 mL), and water (13.3 mL). The mixture was filtered and the filter cake was rinsed with ethyl acetate. The filtrate was concentrated under reduced pressure to provide the title compound. MS (DCI/NH₃) m/z: 203 (M+H)⁺.

### EXAMPLE 23G

### 8-benzyl-3-(trifluoroacetyl)-3,8-diazabicyclo[3.2.1]octane

8-Benzyl-3,8-diazabicyclo[3.2.1]octane (5.18 g, 25.6 mmol) and triethylamine (17.8 mL, 128 mmol) were combined in dry dichloromethane (50 mL) at 0° C., treated with trifluoroacetic anhydride in 1 mL portions (8.9 mL, 64 mmol), and stirred overnight at room temperature. The mixture was concentrated under reduced pressure and the residue was purified by chromatography on silica gel (9:1 hexanes:ethyl acetate) to provide the title compound. MS (DCI/NH₃) m/z: 299 (M+H)⁺; ¹H NMR (CDCl₃) δ 1.66 (m, 2H), 2.06 (br, 2H), 3.08 (d, 1H), 3.25 (d, 2H), 3.53 (m, 4H), 4.12 (d, 1H), 7.34 (m, 5H).

### EXAMPLE 23H

### tert-butyl 3-(trifluoroacetyl)-3,8-diazabicyclo[3.2.1]octane-8-carboxylate

8-Benzyl-3-(trifluoroacetyl)-3,8-diazabicyclo[3.2.1]octane (1.95 g, 6.54 mmol) and di-tert-butyl dicarbonate (2.14 g, 9.81 mmol) were combined in ethyl acetate (75 mL) and treated with Pearlman's catalyst (216 mg) under a hydrogen atmosphere (1 atmosphere) with stirring for 48 hours at room temperature. The mixture was filtered and the filtrate was concentrated under reduced pressure. The residue in ethyl acetate (150 mL) was treated with L-aspartic acid and stirred for 2 hours. The mixture was washed with saturated sodium bicarbonate solution and concentrated under reduced pressure to provide the title compound. ¹H NMR (CDCl₃) δ 1.48 (s, 9H), 1.70 (m, 2H), 1.98 (m, 2H), 3.06 (d, 1H), 3.45 (d, 1H), 3.70 (d, 1H), 4.23 (d, 1H), 4.30 (br, 2H).

### EXAMPLE 23I

### tert-butyl 3,8-diazabicyclo[3.2.1]octane-8-carboxylate

tert-Butyl 3-(trifluoroacetyl)-3,8-diazabicyclo[3.2.1]octane-8-carboxylate (6.54 mmol) in methanol (85 mL) was treated with ammonium hydroxide (8.5 mL) and stirred overnight at room temperature. The mixture was concentrated under reduced pressure and purified by chromatography on silica gel (5% MeOH, 0.5% NH₄OH in CH₂Cl₂; then 10% MeOH, 1% NH₄OH in CH₂Cl₂) to provide the title compound. MS (DCI/NH₃) m/z: 213 (M+H)⁺; ¹H NMR (CDCl₃) δ 1.47 (s, 9H), 1.81 (m, 2H), 1.94 (m, 2H), 2.65 (dd, 2H), 3.00 (br, 2H), 4.11 (br, 2H).

### EXAMPLE 23J

### tert-butyl 3-(3-chloro-2-pyridinyl)-3,8-diazabicyclo[3.2.1]octane-8-carboxylate

tert-Butyl 3,8-diazabicyclo[3.2.1]octane-8-carboxylate (435 mg, 2.26 mmol), 2-bromo-3-chloropyridine (400 mg, 1.88 mmol), and potassium carbonate (390 mg, 2.83 mmol) were combined in dry dimethylformamide (10 mL) and stirred at 120° C. for 48 hours. The mixture was allowed to cool room temperature, diluted with ethyl acetate, filtered, and the filtrate was concentrated under reduced pressure. The residue was purified by chromatography on silica gel (85:15 hexanes:ethyl acetate) to provide the title compound. MS (ESI) m/z: 268 (100%), 324.0 (M+H)⁺; ¹H NMR (CDCl₃) δ 1.48 (s, 9H), 1.92 (m, 2H), 2.07 (m, 2H), 3.13 (d, 2H), 3.59 (br, 2H), 4.30 (br, 2H), 6.84 (dd, 1H), 7.58 (dd, 1H), 8.17 (dd, 1H).

### EXAMPLE 23K

### 3-(3-chloro-2-pyridinyl)-3,8-diazabicyclo[3.2.1]octane

tert-Butyl 3-(3-chloro-2-pyridinyl)-3,8-diazabicyclo[3.2.1]octane-8-carboxylate (419 mg, 1.29 mmol) in dry dichloromethane (5 mL) was treated with trifluoroacetic acid and stirred at room temperature for an hour. The mixture was concentrated under reduced pressure and the residue was treated with IN sodium hydroxide solution. The mixture was extracted with ethyl acetate and the organic phase was concentrated under reduced pressure to provide the title compound. MS (ESI) m/z: 224 (M+H)⁺; ¹H NMR (CDCl₃) δ 1.86 (m, 2H), 2.09 (m, 2H), 3.09 (d, 2H), 3.63 (m, 4H), 6.81 (dd, 1H), 7.56 (dd, 1H), 8.16 (dd, 1H).

### EXAMPLE 23L

### N-(4-tert-butylphenyl)-3-(3-chloro-2-pyridinyl)-3,8-diazabicyclo[3.2.1]octane-8-carboxamide

3-(3-Chloro-2-pyridinyl)-3,8-diazabicyclo[3.2.1]octane (129 mg, 577 μmol) in dry dichloromethane (5 mL) was treated with 1-tert-butyl-4-isocyanatobenzene (101 mg, 577 μmol) and allowed to stir overnight at room temperature. The mixture was concentrated under reduced pressure and the residue was purified by chromatography on silica gel (3:1, hexanes:ethyl acetate) to provide the title compound. MS (DCI/NH₃) m/z: 399 (M+H)⁺; 1H NMR (CDCl₃) δ 1.30 (s, 9H), 2.01 (m, 2H), 2.17 (m, 2H), 3.25 (d, 2H), 3.64 (dd, 2H), 4.38 (dd, 2H), 6.32 (s, 1H), 6.86 (dd, 1H), 7.32 (s, 4H), 7.59 (dd, 1H), 8.17 (dd, 1H).

### EXAMPLE 24

### 3-(3-chloro-2-pyridinyl)-N-(3,4-dichlorophenyl)-3,8-diazabicyclo[3.2.1]octane-8-carboxamide

The title compound was prepared using the procedure described in Example 23L using 1,2-dichloro-4-isocyanatobenzene instead of 1-tert-butyl-4-isocyanatobenzene. MS (ESI) m/z: 413 (M+H)⁺; ¹H NMR (DMSO-d₆) δ 1.87 (m, 2H), 2.01 (m, 2H), 3.06 (d, 2H), 3.55 (dd, 2H), 4.53 (s, 2H), 7.01 (dd, 1H), 7.47 (m, 2H), 7.79 (dd, 1H), 7.90 (dd, 1H), 8.21 (dd, 1H), 8.90 (s, 1H).

### EXAMPLE 25

### 3-(3-chloro-2-pyridinyl)-N-[3-(trifluoromethyl)phenyl]-3,8-diazabicyclo[3.2.1]octane-8-carboxamide

The title compound was prepared using the procedure described in Example 23L using 1-isocyanato-3-(trifluoromethyl)benzene instead of 1-tert-butyl-4-isocyanatobenzene. MS (ESI) m/z: 411 (M+H)⁺; ¹H NMR (DMSO-d₆) δ 1.88 (m, 2H), 2.02 (m, 2H), 3.07 (d, 2H), 3.53 (dd, 2H), 4.55 (dd, 2H), 7.00 (dd, 1H), 7.27 (dd, 1H), 7.48 (t, 1H), 7.80 (dd, 1H), 7.99 (t, 1H), 8.21 (dd, 1H), 8.95 (s, 1H).

### EXAMPLE 26

### 3-(3-chloro-2-pyridinyl)-N-[4-(trifluoromethyl)phenyll-3,8-diazabicyclo[3.2.1]octane-8-carboxamide

The title compound was prepared using the procedure described in Example 23L using 1-isocyanato-4-(trifluoromethyl)benzene instead of 1-tert-butyl-4-isocyanatobenzene. MS (ESI) m/z: 411 (M+H)⁺; ¹H NMR (DMSO-d₆) δ 1.88 (m, 2H), 2.02 (m, 2H), 3.07 (d, 2H), 3.55 (dd, 2H), 4.56 (dd, 2H), 7.00 (dd, 1H), 7.60 (d, 2H), 7.75 (d, 2H), 7.80 (dd, 1H), 8.21 (dd, 1H), 9.00 (s, 1H).

### EXAMPLE 27

### 3-(3-chloro-2-pyridinyl)-N-(4-fluorophenyl)-3,8-diazabicyclo[3.2.1]octane-8-carboxamide

The title compound was prepared using the procedure described in Example 23L using 1-fluoro-4-isocyanatobenzene instead of 1-tert-butyl-4-isocyanatobenzene. MS (ESI) m/z: 361 (M+H)⁺; ¹H NMR (DMSO-d₆) δ 1.86 (m, 2H), 2.00 (m, 2H), 3.06 (d, 2H), 3.53 (dd, 2H), 4.51 (dd, 2H), 7.01 (dd, 1H), 7.08 (t, 2H), 7.51 (dd, 2H), 7.79 (dd, 1H), 8.21 (dd, 1H), 8.65 (s, 1H).

### EXAMPLE 28

### N-(4-chlorophenyl)-3-(3-chloro-2-pyridinyl)-3,8-diazabicyclo[3.2.1]octane-8-carboxamide

The title compound was prepared using the procedure described in Example 23L using 1-chloro-4-isocyanatobenzene instead of 1-tert-butyl-4-isocyanatobenzene. MS (ESI) m/z: 377 (M+H)⁺; ¹H NMR (DMSO-d₆) δ 1.86 (m, 2H), 2.00 (m, 2H), 3.06 (d, 2H), 3.54 (dd, 2H), 4.52 (s, 2H), 7.01 (dd, 1H), 7.29 (d, 2H), 7.55 (d, 2H), 7.79 (dd, 1H), 8.21 (dd, 1H), 8.74 (s, 1H).

### EXAMPLE 29

### N-(4-bromophenyl)-3-(3-chloro-2-pyridinyl)-3,8-diazabicyclo[3.2.1]octane-8-carboxamide

The title compound was prepared using the procedure described in Example 23L using 1-bromo-4-isocyanatobenzene instead of 1-tert-butyl-4-isocyanatobenzene. MS (ESI) m/z: 423 (M+H)⁺; IH NMR (DMSO-d₆) δ 1.86 (m, 2H), 2.00 (m, 2H), 3.06 (d, 2H), 3.54 (dd, 2H), 4.53 (s, 2H), 7.01 (dd, 1H), 7.41 (d, 2H), 7.51 (d, 2H), 7.79 (dd, 1H), 8.21 (dd, 1H), 8.75 (s, 1H).

### EXAMPLE 30

### 3-(3-chloro-2-pyridinyl)-N-(4-iodophenyl)-3,8-diazabicyclo[3.2.1]octane-8-carboxamide

The title compound was prepared using the procedure described in Example 23L using 1-iodo-4-isocyanatobenzene instead of 1-tert-butyl-4-isocyanatobenzene. MS (ESI) m/z: 469 (M+H)⁺; ¹H NMR (DMSO-d₆) δ 1.86 (m, 2H), 2.00 (m, 2H), 3.05 (d, 2H), 3.53 (dd, 2H), 4.52 (d, 2H), 7.01 (dd, 1H), 7.38 (d, 2H), 7.56 (d, 2H), 7.79 (dd, 1H), 8.21 (dd, 1H), 8.72 (s, 1H).

### EXAMPLE 31

### N-(4-butylphenyl)-3-(3-chloro-2-pyridinyl)-3,8-diazabicyclo[3.2.1]octane-8-carboxamide

The title compound was prepared using the procedure described in Example 23L using 1-butyl-4-isocyanatobenzene instead of 1-tert-butyl-4-isocyanatobenzene. MS (ESI) m/z: 399 (M+H)⁺; ¹H NMR (DMSO-d₆) δ 0.89 (t, 3H), 1.29 (6, 2H), 1.52 (5,2H), 1.86 (m, 2H), 1.99 (m, 2H), 2.50 (t, 2H), 3.06 (d, 2H), 3.52 (dd, 2H), 4.51 (s, 2H), 7.00 (dd,1H), 7.05 (d, 2H), 7.39 (d, 2H), 7.79 (dd, 1H), 8.21 (dd, 1H), 8.52 (s, 1H).

### EXAMPLE 32

### 3-(3-chloro-2-pyridinyl)-N-(4-isopropylphenyl)-3,8-diazabicyclo[3.2.1]octane-8-carboxamide

The title compound was prepared using the procedure described in Example 23L using 1-isocyanato-4-isopropylbenzene instead of 1-tert-butyl-4-isocyanatobenzene. MS (ESI) m/z: 385 (M+H)⁺; ¹H NMR (DMSO-d₆) δ 1.17 (d, 6H), 1.86 (m, 2H), 1.99 (m, 2H), 2.81 (7, 2H), 3.06 (d, 2H), 3.52 (dd, 2H), 4.51 (s, 2H), 7.00 (dd, 1H), 7.11 (d, 2H), 7.41 (d, 2H), 7.79 (dd, 1H), 8.21 (dd, 1H), 8.54 (s, 1H).

### EXAMPLE 33

### 3-(3-chloro-2-pyridinyl)-N-{4-[(trifluoromethyl)thio]phenyl}-3,8-diazabicyclo[3.2.1]octane -8-carboxamide

The title compound was prepared using the procedure described in Example 23L using 1-isocyanato-4-[(trifluoromethyl)thio]benzene instead of 1-tert-butyl-4-isocyanatobenzene. MS (ESI) m/z: 443 (M+H)⁺; ¹H NMR (DMSO-d₆) δ 1.88 (m, 2H), 2.01 (m, 2H), 3.07 (d, 2H), 3.55 (dd, 2H), 4.55 (s, 2H), 7.01 (dd, 1H), 7.58 (d, 2H), 7.70 (d, 2H), 7.80 (dd, 1H), 8.21 (dd, 1H), 8.98 (s, 1H).

### EXAMPLE 34

### N-(4-tert-butylphenyl)-8-(3-chloro-2-pyridinyl)-3,8-diazabicyclo[3.2.1]octane-3-carboxamide

### EXAMPLE 34A

### 8-benzyl-3,8-diazabicyclo[3.2.1]octane

tert-Butyl 3-(trifluoroacetyl)-3,8-diazabicyclo[3.2.1]octane-8-carboxylate (1.63 g, 5.46 mmol) in methanol (50 mL) was treated with ammonium hydroxide (7.5 mL) and stirred overnight at room temperature.

### EXAMPLE 34B

### tert-butyl 8-benzyl-3,8-diazabicyclo[3.2.1]octane-3-carboxylate

8-Benzyl-3,8-diazabicyclo[3.2.1]octane (5.46 mmol) and di-tert-butyl dicarbonate (1.79 g, 8.20 mmol) were combined in ethyl acetate (25 mL) and stirred overnight at room temperature. The mixture was concentrated under reduced pressure, dissolved in ethyl acetate(150 mL), treated with L-aspartic acid (2.18 g, 16.4 mmol), and stirred overnight. The mixture was washed with saturated sodium bicarbonate solution and concentrated under reduced pressue to provide the title compound.

### EXAMPLE 34C

### tert-butyl 3,8-diazabicyclo[3.2.1]octane-3-carboxylate

tert-Butyl 8-benzyl-3,8-diazabicyclo[3.2.1]octane-3-carboxylate (5.46 mmol, 203 mg) and Pearlman's catalyst were combined in methanol under a hydrogen atmosphere (1 atmosphere) stirred overnight at room temperature. Purged with nitrogen, filtered off the catalyst, and concentrated the filtrate to a yellow liquid. The mixture was filtered and the filtrate was concentrated under reduced pressure. The residue was purified by chromatography on silica gel (5% MeOH, 0.5% NH₄OH in CH₂Cl₂, then 10% MeOH, 1% NH₄OH in CH₂Cl₂) to provide the title compound. MS (DCI/NH₃) m/z: 213 (M+H)⁺; ¹H NMR (CDCl₃) δ 1.45 (s, 9H), 1.75 (br, 4H), 2.93 (d, 1H), 3.01 (d, 1H), 3.47 (d, 2H), 3.68 (d, 1H), 3.81 (d, 1H).

### EXAMPLE 34D

### tert-butyl 8-(3-chloro-2-pyridinyl)-3,8-diazabicyclo[3.2.1]octane-3-carboxylate

tert-Butyl 3,8-diazabicyclo[3.2.1]octane-3-carboxylate (454 mg, 2.36 mmol) and 2-bromo-3-chloropyridine (418 mg, 1.97 mmol) were combined in dry N,N-dimethylformamide (10 mL) and treated with potassium carbonate (408 mg, 2.95 mmol). After stirring at 120° C. for 48 hours, the mixture was allowed to cool to room temperature, diluted with ethyl acetate, filtered, and the filtrate was concentrated under reduced pressure. The residue was purified by chromatography on silica gel (4:1, hexanes:ethyl acetate) to provide the title compound. MS (ESI) m/z: 324 (M+H)⁺; ¹H NMR (CDCl₃) δ 1.47 (s, 9H), 1.77 (m, 2H), 1.93 (m, 2H), 3.22 (d, 1H), 3.31 (d, 1H), 3.76 (d, 1H), 3.90 (d, 1H), 4.53 (d, 2H), 6.76 (dd, 1H), 7.56 (dd, 1H), 8.09 (dd, 1H).

### EXAMPLE 34E

### 8-(3-chloro-2-pyridinyl)-3,8-diazabicyclo[3.2.1]octane

tert-Butyl 8-(3-chloro-2-pyridinyl)-3,8-diazabicyclo[3.2.1]octane-3-carboxylate (285 mg, 880 μmol) in dry dichloromethane (2 mL) was treated with trifluoroacetic acid (2 mL). After stirring at ambient temperature for 30 minutes, the mixture was concentrated under reduced pressure and the residue was treated with 1N sodium hydroxide solution. The mixture was extracted with ethyl acetate and the organic phase was concentrated under reduced pressure to provide the title compound. MS (DCI/NH₃) m/z: 224 (M+H)⁺; ¹H NMR (CDCl₃) δ 1.87 (m, 2H), 1.97 (m, 2H), 2.75 (dd, 2H), 3.24 (dd, 2H), 4.44 (dd, 2H), 6.73 (dd, 1H), 7.54 (dd, 1H), 8.08 (dd, 1H).

### EXAMPLE 34F

### N-(4-tert-butylphenyl)-8-(3-chloro-2-pyridinyl)-3,8-diazabicyclo[3.2.1]octane-3-carboxamide

8-(3-Chloro-2-pyridinyl)-3,8-diazabicyclo[3.2.1]octane (102 mg, 456 μmol) and 1-tert-butyl-4-isocyanatobenzene (80 mg, 456 μmol) were combined in dry dichloromethane (4.5 mL) and stirred overnight at room temperature. The mixture was concentrated and the residue was purified by chromatography on silica gel (7:3, hexanes:ethyl acetate) to provide the title compound. MS (ESI) m/z: 399 (M+H)⁺; ¹H NMR (DMSO-d₆) δ 1.25 (s, 9H), 1.73 (m, 2H), 1.85 (m, 2H), 3.20 (dd, 2H), 3.92 (dd, 2H), 4.51 (dd, 2H), 6.94 (dd, 1H), 7.24 (d, 2H), 7.37 (d, 2H), 7.79 (dd, 1H), 8.17 (dd, 1H), 8.30 (s, 1H).

### EXAMPLE 35

### 8-(3-chloro-2-pyridinyl)-N-[4-(trifluoromethyl)phenyl]-3,8-diazabicyclo[3.2.1]octane-3-carboxamide

The title compound was prepared using the procedure described in Example 34F using 1-isocyanato-4-(trifluoromethyl)benzene instead of 1-tert-butyl-4-isocyanatobenzene. MS (ESI) m/z: 411 (M+H)⁺; ¹H NMR (DMSO-d₆) δ 1.74 (m, 2H), 1.87 (m, 2H), 3.25 (d, 2H), 3.95 (dd, 2H), 4.53 (dd, 2H), 6.95 (dd, 1H), 7.58 (d, 2H), 7.70 (d, 2H), 7.80 (dd, 1H), 8.17 (dd, 1H), 8.78 (s, 1H).

### EXAMPLE 37

### N-(4-tert-butylphenyl)-1-(3-chloro-2-pyridinyl)-(cis)-3-hydroxy-4-piperidinecarboxamide

### EXAMPLE 37A

### ethyl 1-benzyl-3-hydroxy-4-piperidinecarboxylate

Ethyl 1-benzyl-3-oxo-4-piperidinecarboxylate (1.0 g, 3.8 mmol) in diethyl ether (50 mL) was treated with lithium aluminumhydride (150 mg, 3.9 mmol) at and −78° C. After stirring at −78° C. for 30 minutes, the mixture was allowed to warm to room temperature over 2 hours. The mixture was quenched with saturated ammonium chloride. Standard work-up gave a crude product which was purified via chromatography (SiO₂, ethyl acetate:hexanes, 1:6) to provide the title compound. MS (EI, M+H)⁺264.

### EXAMPLE 37B

### ethyl 3-hydroxy-4-piperidinecarboxylate

Ethyl 1-benzyl-3-hydroxy-4-piperidinecarboxylate in ethanol (10 mL) was treated with Pd(OH)₂ (20% on carbon, 50 mg) under a hydrogen atmosphere (1 atm) and stirred for 5 hours. The reaction mixture was filtered and the filtrate was concentrated under reduced pressure.

### EXAMPLE 37C

### ethyl 1-(3-chloro-2-pyridinyl)-3-hydroxy-4-piperidinecarboxylate

Ethyl 3-hydroxy-4-piperidinecarboxylate, 2-bromo-3-chloropyridine (700 mg, 3.6 mmol), and potassium carbonate (1.0 g, 7.2 mmol) were and heated at 90° C. for 2 days. Standard work-up gave the title compound.

### EXAMPLE 37D

### 1-(3-chloro-2-pyridinyl)-3-hydroxy-4-piperidinecarboxylic acid

Ethyl 1-(3-chloro-2-pyridinyl)-3-hydroxy-4-piperidinecarboxylate in water (55 mL) was treated with sodium methoxide (0.5 M, 8 mL, 4.0 mmol) at room temperature. After 2 hours, the mixture was concentrated under reduced pressure and the residue was purified by column chromatography to provide the title compound. MS (EI, M+H)⁺257.

### EXAMPLE 37E

### N-(4-tert-butylphenyl)-1-(3-chloro-2-pyridinyl)-(cis)-3-hydroxy-4-piperidinecarboxamide

1-(3-Chloro-2-pyridinyl)-3-hydroxy-4-piperidinecarboxylic acid, tert-butylanaline (255 mg, 1.7 mmol), and EDCI (380 mg, 2.0 mmol) were combined at room temperature. The mixture was allowed to stir for 8 hours and standard work-up provided crude product which was purified by column chromatography to provide the cis and trans analogues. ¹H NMR (500 MHz, CDCl₃) δ 8.15 (dd, 1H), 7.68 (dd, 1H), 7.46 (m, 2H), 7.32 (m, 2H), 6.95 (dd, 1H), 4.32 (m, 1H), 4.08 (m, 1H), 3.95 (m, 1H), 3.25 (m, 2H), 2.56 (m, 1H), 1.92 (m, 2H), 1.28 (s, 9H); MS (ESI, M+H)⁺388.

### EXAMPLE 38

### N-(4-tert-butylphenyl)-1-(3-chloro-2-pyridinyl)-(trans)-3-hydroxy-4-piperidinecarboxamide

The title compound was isolated from the purification step in Example 37E. ¹H NMR (500 MHz, CDCl₃) δ 8.26 (dd, 1H), 7.96 (s(br), 1H), 7.64 (dd, 1H), 7.45 (m, 2H), 7.34 (m, 2H), 6.86 (dd, 1H), 4.22 (ddd, 1H), 4.05 (m, 1H), 3.96 (m, 1H), 3.02 (m, 1H), 2.94 (m, 1H), 2.42 (m, 1H), 2.14 (m, 1H), 1.96 (m, 1H), 1.30 (s, 9H); MS (ESI, M+H)⁺388.

### EXAMPLE 39

### 1-(3-chloro-2-pyridinyl)-4-hydroxy-N-[4-(trifluoromethyl)phenyl]-4-piperidinecarboxamide

### Example 39A

### methyl 1-benzyl-4-hydroxy-4-piperidinecarboxylate

1-Benzyl-4-hydroxy-4-piperidinecarbonitrile (30.0 g, 118 mmol) in methanol (590 mL) and concentrated HCl (590 mL) was heated at reflux for 18 hours. The product was obtained following chromatography (SiO₂, ethyl acetate:hexane, 1:4). MS (EI, M+H)⁺250.

### EXAMPLE 39B

### methyl 4-hydroxy-4-piperidinecarboxylate

Methyl 1-benzyl-4-hydroxy-4-piperidinecarboxylate in methanol was treated with Pd(OH)₂ (20% on carbon, 500 mg) under a hydrogen atmosphere. The mixture was allowed to stir at room temperature for 12 hours, filtered, and the filtrate was concentrated under reduced pressure.

### EXAMPLE 39C

### methyl 1-(3-chloro-2-pyridinyl)-4-hydroxy-4-piperidinecarboxylate

Methyl l-benzyl-4-hydroxy-4-piperidinecarboxylate (15.0 g, 77 mmol), 2,3-dichloropyridine (12.0 g, 81 mmol), and potassium carbonate (15.0 g, 108.7 mmol) were combined in DMF (200 mL) and heated at 90° C. for 2 days. The mixture was allowed to cool to room temperature, concentrated, and standard work-up provided a residue that was purified via chromatography (SiO₂, ethyl acetate:hexanes, 1:6) to provide the title compound MS (EI, M+H)⁺271.

### EXAMPLE 39D

### 1-(3-chloro-2-pyridinyl)-4-hydroxy-4-piperidinecarboxylic acid

The title compound was prepared using the procedure described in Example 37D substituting methyl 1-(3-chloro-2-pyridinyl)-4-hydroxy-4-piperidinecarboxylate for ethyl 1-(3-chloro-2-pyridinyl)-3-hydroxy-4-piperidinecarboxylate.

### EXAMPLE 39E

### 1-(3-chloro-2-pyridinyl)-4-hydroxy-N-[4-(trifluoromethyl)phenyl]-4-piperidinecarboxamide

1-(3-Chloro-2-pyridinyl)-4-hydroxy-4-piperidinecarboxylic acid, EDCI (525 mg, 2.7 mmol), and 4-(trifluoromethyl)aniline in dichloromethane (10 mL) were combined at room temperature and stirred overnight. Standard work-up gave a crude product which was purified via chromatography (SiO₂, ethyl acetate:hexanes, 1:9). ¹H NMR (500 MHz, CDCl₃) δ 8.90 (s(br), 1H), 8.20 (dd, 1H), 7.74 (d, 2H), 7.61 (m, 3H), 6.86 (dd, 1H), 3.80 (m, 2H), 3.18 (m, 2H), 2.50 (m, 2H), 1.78 (m, 2H); MS (ESI, M+H)⁺400.

### EXAMPLE 40

### N-(4-tert-butylphenyl)-3′-(trifluoromethyl)-3,6-dihydro-2H-1,2′-bipyridine-4-carboxamide

### EXAMPLE 40A

### 1-[3-(trifluoromethyl)pyridin-2-yl]piperidin-4-one

2-Chloro-3-(trifluoromethyl)pyridine (11.92 g, 65.7 mmol)), K₂CO₃(19.10 g, 138 mmol), and 1,4-dioxa-8-azaspiro[4.5]decane (8.85 mL, 69.0 mmol) were combined in DMSO (65 mL) and stirred at 100° C. for 3 hours. The mixture was treated with additional 1,4-dioxa-8-azaspiro[4.5]decane (2.0 mL, 16 mmol), stirred for 2 hours, treated with additional 1,4-dioxa-8-azaspiro[4.5]decane (1.0 mL, 7.8 mmol), and stirred for 1 hour. The mixture was diluted with diethyl ether (200 mL), washed with water (250 mL), washed with brine (100 mL), dried (Na₂SO₄), filtered, and the filtrate was concentrated under reduced pressure. The residue was dissolved in concentrated HCl (25 mL), stirred for 3 hours, basified with concentrated NH₄OH, extracted with CH₂Cl₂, and the phases separated. The organic phase was dried (Na₂SO₄), filtered, and the filtrate concentrated under reduced pressure. The residue was purified via flash chromatography (20% to 40% diethyl ether/hexanes) to provide the title compound.

### EXAMPLE 40B

### methyl 3′-(trifluoromethyl)-3,6-dihydro-2H-1,2′-bipyridine-4-carboxylate

Lithium diisopropyl amide (37.3 mmol) in THF (75 mL) at −78° C. was treated with 1-[3-(trifluoromethyl)pyridin-2-yl]piperidin-4-one (8.30 g, 34.0 mmol) in THF (25 mL) and stirred for 45 minutes. The mixture was treated with solid PhNTf₂ (14.0 g, 39.1 mmol), stirred for 1 hour, and concentrated under reduced pressure. The residue was diluted with ethyl acetate:hexanes (1:1), washed with 1N NaOH, dried (Na₂SO₄), filtered through a silica plug, and the filtrate was concentrated under reduced pressure. The residue, triethylamine (14.2 mL, 102 mmol), and PdCl₂(PPh₃)₂ (0.944 g, 1.34 mmol) were combined in methanol (7.00 mL, 173 mmol) and DMF (100 mL) and saturated with carbon monoxide gas (bubbling 15 minutes). The mixture was heated at 80° C. under a carbon monoxide atmosphere (1 atm) overnight. The mixture was concentrated to half volume, diluted with diethyl ether, washed with water, brine, dried (Na₂SO₄), filtered, and the filtrate was concentrated under reduced pressure. The residue was purified by flash chromatography (6% ethyl acetate/hexanes) to provide the title compound.

### EXAMPLE 40C

### 3′-(trifluoromethyl)-3,6-dihydro-2H-1,2′-bipyridine-4-carboxylic acid

Methyl 3′-(trifluoromethyl)-3,6-dihydro-2H-1,2′-bipyridine-4-carboxylate (15 mL) in THF (30 mL) was treated with 1N NaOH (27 mL) and stirred for 2 hours. The mixture was treated with additional 1N NaOH (16 mL), stirred for 1 hour, treated with 1N NaOH (14.5 mL), and stirred for 1 hour. The mixture was diluted with water and extracted with CH₂Cl₂. The aqueous layer was then acidified with concentrated HCl and extracted with CHCl₃. The organic layer was dried (Na₂SO₄), filtered, and the filtrate was concentrated under reduced pressure to provide the title compound.

### EXAMPLE 40D

### N-(4-tert-butylphenyl)-3′-(trifluoromethyl)-3,6-dihydro-2H-1,2′-bipyridine-4-carboxamide

3′-(Trifluoromethyl)-3,6-dihydro-2H-1,2′-bipyridine-4-carboxylic acid (0.300 g, 1.10 mmol) and a catalytic amount of DMF were combined in CH₂Cl₂ (4.0 mL) and treated with (COCl)₂ (0.14 mL, 1.6 mmol). The mixture was stirred for 90 minutes, diluted with toluene (0.5 mL) and concentrated to dryness. The residue was dissolved in CH₂Cl₂ (4.0 mL) treated with pyridine (0.14 mL, 1.7 mmol), a catalytic amount of DMAP, and 4-tert-butylaniline (0.21 mL, 1.3 mmol). After 1 hour, the mixture was diluted with water and extracted with CH₂Cl₂. The organic phase was dried (Na₂SO₄), filtered, and the filtrate was concentrated under reduced pressure. The residue was purified by flash chromatography (2.5% ethyl acetate/CH₂Cl₂) to provide the title compound. ¹H NMR (300 MHz, CDCl₃) δ 8.42 (dd, 1H), 7.89 (dd, 1H), 7.47 (d, 3H), 6.98 (dd, 1H), 6.76 (s, 1H), 4.06 (q, 2H), 3.53 (t, 2H), 2.67 (m, 2H); MS (m/z) 404.

### EXAMPLE 41

### N-(4-chlorophenyl)-3′-(trifluoromethyl)-3,6-dihydro-2H-1,2′-bipyridine-4-carboxamide

The title compound was prepared using the procedure described in Example 40D using 4-chloroaniline instead of 4-tert-butylaniline. 1H NMR (300 MHz, CDCl₃) δ 8.42 (dd, 1H), 7.89 (dd, 1H), 7.51 (dd, 2H), 7.43 (brs, 1H), 7.30 (d, 2H), 6.99 (dd, 1H), 6.77 (sept, 1H), 4.05 (q, 2H), 3.51 (t, 2H), 2.65 (m, 2H); MS (m/z) 382.

### EXAMPLE 42

### N-[4-(trifluoromethoxy)phenyl]-3′-(trifluoromethyl)-3,6-dihydro-2H-1,2′-bipyridine-4-carboxamide

The title compound was prepared using the procedure described in Example 40D using 4-(trifluoromethoxy)aniline instead of 4-tert-butylaniline. ¹H NMR (300 MHz, CDCl₃) δ 8.44 (dd, 1H), 7.90 (dd, 1H), 7.59 (d, 2H), 7.48 (brs, 1H), 7.20 (d, 2H), 7.00 (dd, 1H), 6.79 (m, 1H), 4.07 (q, 2H), 3.53 (t, 2H), 2.67 (m, 2H); MS (m/z) 432.

### EXAMPLE 43

### 3′-(trifluoromethyl)-N-{4-[(trifluoromethyl)thio]phenyl}-3,6-dihydro-2H-1,2′-bipyridine -4-carboxamide

The title compound was prepared using the procedure described in Example 40D using 4-[(trifluoromethyl)thio]aniline instead of 4-tert-butylaniline. ¹H NMR (300 MHz, CDCl₃) δ 8.43 (dd, 1H), 7.90 (dd, 1H), 7.66 (d, 2H), 7.62 (d, 2H), 7.53 (brs, 1H), 7.00 (dd, 1H), 6.79 (m, 1H), 4.07 (q, 2H), 3.52 (t, 2H), 2.67 (m, 2H); MS (m/z) 448.

### EXAMPLE 44

### 3′-(trifluoromethyl)-N-{4-[(trifluoromethyl)sulfonyl]phenyl}-3,6-dihydro-2H-1,2′-bipyridine -4-carboxamide

The title compound was prepared using the procedure described in Example 40D using 4-[(trifluoromethyl)sulfonyl]aniline instead of 4-tert-butylaniline. 1H NMR (300 MHz, CDCl₃) δ 8.43 (dd, 1H), 8.00 (dd, 1H), 7.88-7.92 (m, 3H), 7.75 (brs, 1H), 7.02 (dd,1H), 6.85 (m, 1H), 4.09 (q, 2H), 3.52 (t, 2H), 2.68 (m, 2H); MS (m/z) 480.

### EXAMPLE 45

### N-(3-fluoro-4-methylphenyl)-3′-(trifluoromethyl)-3,6-dihydro-2H-1,2′-bipyridine-4-carboxamide

The title compound was prepared using the procedure described in Example 40D using 3-fluoro-4-methylaniline instead of 4-tert-butylaniline. ¹H NMR (300 MHz, CDCl₃) δ 8.42 (dd, 1H), 7.88 (dd, 1H), 7.47 (dd, 1H), 7.40 (brs, 1H), 7.05-7.15 (m, 2H), 6.98 (dd, 1H), 6.76 (sept, 1H), 4.05 (q, 2H), 3.51 (t, 2H), 2.66 (m, 2H); MS (m/z) 380.

### EXAMPLE 46

### N-(4-chlorophenyl)-1-pyrimidin-2-yl-1,2,3,6-tetrahydropyridine-4-carboxamide

### EXAMPLE 46A

### 1-pyrimidin-2-yl-1,2,3,6-tetrahydropyridine-4-carboxylic acid

2-Chloropyrimidine (0.570 g, 4.98 mmol) and 1,2,3,6-tetrahydro-4-pyridinecarboxylic acid (1.22 g) were combined in water (7 mL) and heated at 90° C. After 4 hours, the mixture was treated with additional 1,2,3,6-tetrahydro-4-pyridinecarboxylic acid (0.52 g) and stirred overnight. The mixture was diluted with water and extracted with CH₂Cl₂. The aqueous layer was acidified with concentrated HCl (pH˜3) and extracted with CHCl₃. The organic layer was dried (Na₂SO₄), filtered, and the filtrate was concentrated under reduced pressure to provide the title.

### EXAMPLE 46B

### N-(4-chlorophenyl)-1-pyrimidin-2-yl-1,2,3,6-tetrahydropyridine-4-carboxamide

1-Pyrimidin-2-yl-1,2,3,6-tetrahydropyridine-4-carboxylic acid (55.0 mg, 0.27 mmol) and a catalytic amount of DMF were combined in CH₂Cl₂ (1.5 mL) and treated with (COCl)₂ (0.033 mL, 0.38 mmol). The mixture was stirred for 90 minutes, diluted with toluene (0.5 mL), and concentrated to dryness under reduced pressure. The residue in CH₂Cl₂ (1.5 mL) was treated with pyridine (0.033 mL, 0.41 mmol), catalytic amount of DMAP, and 4-chloroaniline (41.0 mg, 0.32 mmol). The mixture was stirred for 1 hour, diluted with water, and extracted with CH₂Cl₂. The organic phase was dried (Na₂SO₄), filtered, and the filtrate was concentrated under reduced pressure. The residue was purified by flash chromatography (15% ethyl acetate/CH₂Cl1₂) to provide the title compound. ¹H NMR (300 MHz, CDCl₃) δ 8.35 (d, 2H), 7.51 (d, 2H), 7.39 (brs, 1H), 7.31 (d, 2H), 6.77 (sept, 1H), 6.54 (t, 1H), 4.43 (q, 2H), 4.03 (t, 2H), 2.58 (m, 2H); MS (m/z) 315.

### EXAMPLE 47

### 1-pyrimidin-2-yl-N-{4-[(trifluoromethyl)thio]phenyl}-1,2,3,6-tetrahydropyridine-4-carboxamide

The title compound was prepared using the procedure described in Example 46B using 4-[(trifluoromethyl)thio]aniline instead of 4-chloroaniline. ¹H NMR (300 MHz, CDCl₃) δ 8.35 (d, 2H), 7.64 (s, 4H), 7.50 brs, 1H), 6.78 (sept, 1H), 6.56 (t, 1H), 4.45 (q, 2H), 4.05 (t, 2H), 2.59 (m, 2H); MS (m/z) 381.

### EXAMPLE 48

### 1-pyrimidin-2-yl-N-{4-[(trifluoromethyl)sulfonyl]phenyl}-1,2,3,6-tetrahydropyridine-4-carboxamide

The title compound was prepared using the procedure described in Example 46B using 4-[(trifluoromethyl)sulfonyl]aniline instead of 4-chloroaniline. ¹H NMR (300 MHz, CDCl₃) δ 8.36 (d, 2H), 8.00 (d, 2H), 7.90 (d, 2H), 7.72 (brs, 1H), 6.84 (m, 1H), 6.57 (t, 1H), 4.48 (q, 2H), 4.06 (t, 2H), 2.61 (m, 2H); MS (m/z) 413.

The foregoing is merely illustrative and is not intended to limit the invention to the disclosed compounds. Variations and changes which are obvious to one skilled in the art are to be within the scope and nature of the invention which are defined in the appended claims.

### In Vivo Data

Determination of Antinociceptive Effect

Experiments were performed on 400 adult male 129J mice (Jackson laboratories, Bar Harbor, Me.), weighing 20-25 g. Mice were kept in a vivarium, maintained at 22° C., with a 12 hour alternating light-dark cycle with food and water available ad libitum. All experiments were performed during the light cycle. Animals were randomly divided into separate groups of 10 mice each. Each animal was used in one experiment only and was sacrificed immediately following the completion of the experiment. All animal handling and experimental procedures were approved by an IACUC Committee.

The antinociceptive test used was a modification of the abdominal constriction assay described in Collier, et al., Br. J. Pharmacol. Chemother. 32 (1968) 295-310. Each animal received an intraperitoneal (i.p.) injection of 0.3 mL of 0.6% acetic acid in normal saline to evoke writhing. Animals were placed separately under clear cylinders for the observation and quantification of abdominal constriction. Abdominal constriction was defined as a mild constriction and elongation passing caudally along the abdominal wall, accompanied by a slight twisting of the trunk and followed by bilateral extension of the hind limbs. The total number of abdominal constrictions was recorded from 5 to 20 minutes after acetic acid injection. The ED_{50S} were determined based on the i.p. injection.

The compounds of the present invention were found to have antinociceptive effects with ED_{50S}, from about 0.1 mg/kg to about 150 mg/kg. The data demonstrates that compounds of the present invention are useful for treating pain.

The present invention also provides pharmaceutical compositions that comprise compounds of the present invention. The pharmaceutical compositions comprise compounds of the present invention formulated together with one or more non-toxic pharmaceutically acceptable carriers.

The pharmaceutical compositions of this invention can be administered to humans and other mammals orally, rectally, parenterally , intracisternally, intravaginally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable carrier or excipient, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of this invention with suitable non-irritating carriers or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a compound of this invention include powders, sprays, ointments and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated.

When used in the above or other treatments, a therapeutically effective amount of one of the compounds of the present invention can be employed in pure form or, where such forms exist, in pharmaceutically acceptable salt, ester or prodrug form. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The term “pharmaceutically acceptable salt,” as used herein, means salts derived from inorganic or organic acids. The salts can be prepared in situ during the final isolation and purification of compounds of formula (I-IV) or separately by reacting the free base of a compound of formula (I-IV) with an inorganic or organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, dihydrochloride, hydrobromide, hydroiodide, 2-hydroxyethansulfonate (isethionate), lactate, maleate, fumarate, methanesulfonate, nicotinate, 2-naphthalenesulfonate, oxalate, pamoate, pectinate, persulfate, 3-phenylpropionate, picrate, pivalate, propionate, succinate, sulfate, (L) tartrate, (D) tartrate, (DL) tartrate, thiocyanate, phosphate, glutamate, bicarbonate, p-toluenesulfonate, and undecanoate.

The term “pharmaceutically acceptable ester,” as used herein, means esters of compounds of the present invention which hydrolyze in vivo and include those that break down readily in the human body to leave the parent compound or a salt thereof. Examples of pharmaceutically acceptable, non-toxic esters of the present invention include C₁-to-C₆ alkyl esters and C₅-to-C₇ cycloalkyl esters, although C₁-to-C₄ alkyl esters are preferred. Esters of the compounds of formula (I-IV) may be prepared according to conventional methods.

The term “pharmaceutically acceptable amide,” as used herein, means to non-toxic amides of the present invention derived from ammonia, primary C₁-to-C₆ alkyl amines and secondary C₁-to-C₆ dialkyl amines. In the case of secondary amines, the amine may also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula (I-IV) may be prepared according to conventional methods.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like. Prodrugs of the present invention may be rapidly transformed in vivo to compounds of formula (I-IV), for example, by hydrolysis in blood.

The present invention contemplates compounds of formula (I-IV) formed by synthetic means or formed by in vivo biotransformation.

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The total daily dose of the compounds of this invention administered to a human or lower animal may range from about 0.01 to about 150 mg/kg/day. For purposes of oral administration, more preferable doses can be in the range of from about 0.1 to about 150 mg/kg/day. If desired, the effective daily dose can be divided into multiple doses for purposes of administration; consequently, single dose compositions may contain such amounts or submultiples thereof to make up the daily dose.

1. A compound of formula (I) 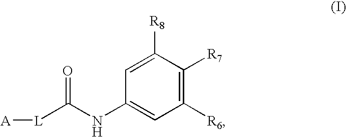
or a pharmaceutically acceptable salt or prodrug thereof, wherein
A is 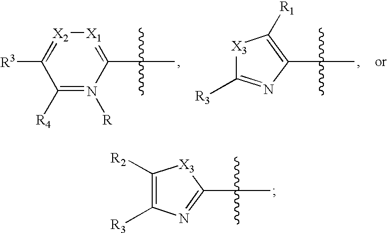
X₁ is N or CR₁;
X₂ is N or CR₂;
X₃ is O or N;
R is absent or O;
R₁ is hydrogen, lower alkoxy, lower alkenyl, lower alkyl, lower alkylthio, lower alkynyl, lower haloalkoxy, lower haloalkyl, lower haloalkylthio, halogen, hydroxy, mercapto, nitro, or —NR_{A}R_{B};
R₂, R₃, and R₄ are independently hydrogen or halogen;
R₇ is hydrogen, alkenyl, alkoxy, alkoxycarbonyl, alkoxysulfonyl, alkyl, alkylcarbonyl, alkylsulfonyl, alkylthio, alkynyl, aryl, arylalkyl, aryloxy, arylthio, cycloalkyl, cycloalkylalkyl, cycloalkyloxy, cycloalkylthio, haloalkoxy, haloalkyl, haloalkylsulfonyl, haloalkylthio, halogen, heteroaryl, heteroarylalkyl, heteroaryloxy, heteroarylthio, heterocycle, heterocyclealkyl, hydroxy, hydroxyalkyl, —NR_{C}R_{D}, (NR_{A}R_{B})carbonyl, or (NR_{A}R_{B})sulfonyl;
R₆ and R₈ are independently hydrogen, lower alkenyl, lower alkoxy, lower alkyl, lower alkylthio, lower alkynyl, lower haloalkoxy, lower haloalkyl, lower haloalkylthio, halogen, hydroxy, mercapto, or —NR_{A}R_{B};
R_{A} and R_{B} are independently hydrogen or alkyl;
R_{C} and R_{D} are independently hydrogen, alkenyl, alkoxycarbonyl, alkyl, alkylcarbonyl, alkynyl, or (NR_{A}R_{B})carbonyl;
L is 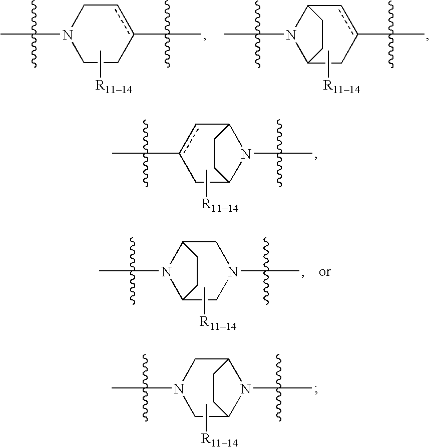
- - is absent or a single bond; and
R₁₁, R₁₂, R₁₃, and R₁₄ are independently hydrogen, alkoxy, alkyl, or hydroxy.

2. The compound according to claim 1 of formula (II) 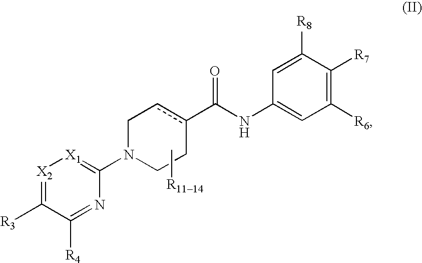
or a pharmaceutically acceptable salt or prodrug thereof.

3. The compound according to claim 2 wherein
- - - is a single bond;
X₁ is CR₁;
X₂ is CR₂;
R₁ is lower haloalkyl or halogen;
R₂, R₃, and R₄ are hydrogen;
R₇ is alkoxy, alkyl, alkylthio, haloalkoxy, haloalkyl, haloalkylsulfonyl, haloalkylthio, halogen, or —NR_{C}R_{D};
R₁₁, R₁₂, R₁₃, and R₁₄ are hydrogen; and
R_{C} and R_{D} are independently hydrogen or alkyl.

4. The compound according to claim 3 that is
N-(4-tert-butylphenyl)-3′-chloro-3,6-dihydro-2H-1,2′-bipyridine-4-carboxamide;
3′-chloro-N-(4-methylphenyl)-3,6-dihydro-2H-1,2′-bipyridine-4-carboxamide;
3′-chloro-N-(4-methoxyphenyl)-3,6-dihydro-2H-1,2′-bipyridine-4-carboxamide;
3′-chloro-N-(4-fluorophenyl)-3,6-dihydro-2H-1,2′-bipyridine-4-carboxamide;
N-(4-bromophenyl)-3′-chloro-3,6-dihydro-2H-1,2′-bipyridine-4-carboxamide;
3′-chloro-N-[4-(trifluoromethoxy)phenyl]-3,6-dihydro-2H-1,2′-bipyridine-4-carboxamide;
3′-chloro-N-(4-ethylphenyl)-3,6-dihydro-2H-1,2′-bipyridine-4-carboxamide;
3′-chloro-N-(4-isopropylphenyl)-3,6-dihydro-2H-1,2′-bipyridine-4-carboxamide;
3′-chloro-N-(4-propoxyphenyl)-3,6-dihydro-2H-1,2′-bipyridine-4-carboxamide;
3′-chloro-N-[4-(methylthio)phenyl]-3,6-dihydro-2H-1,2′-bipyridine-4-carboxamide;
3′-chloro-N-(3-fluoro-4-methylphenyl)-3,6-dihydro-2H-1,2′-bipyridine-4-carboxamide;
3′-chloro-N-[4-(trifluoromethyl)phenyl]-3,6-dihydro-2H-1,2′-bipyridine-4-carboxamide;
3′-chloro-N-[4-(dimethylamino)phenyl]-3,6-dihydro-2H-1,2′-bipyridine-4-carboxamide;
3′-chloro-N-[4-(diethylamino)phenyl]-3,6-dihydro-2H-1,2′-bipyridine-4-carboxamide;
N-(4-tert-butylphenyl)-3′-(trifluoromethyl)-3,6-dihydro-2H-1,2′-bipyridine-4-carboxamide;
N-(4-chlorophenyl)-3′-(trifluoromethyl)-3,6-dihydro-2H-1,2′-bipyridine-4-carboxamide;
N-[4-(trifluoromethoxy)phenyl]-3′-(trifluoromethyl)-3,6-dihydro-2H-1,2′-bipyridine-4-carboxamide;
3′-(trifluoromethyl)-N-{4-[(trifluoromethyl)thio]phenyl}-3,6-dihydro-2H-1,2′bipyridine-4-carboxamide;
3′-(trifluoromethyl)-N-{4-[(trifluoromethyl)sulfonyl]phenyl}-3,6-dihydro-2H-1,2′-bipyridine -4-carboxamide; or
N-(3-fluoro-4-methylphenyl)-3′-(trifluoromethyl)-3,6-dihydro-2H-1,2′-bipyridine-4-carboxamide.

5. The compound according to claim 3 that is 3′-chloro-N-(4-chlorophenyl)-3,6-dihydro-2H-1,2′-bipyridine-4-carboxamide.

6. The compound according to claim 2 wherein
- - - is a single bond;
X₁ is CR₁;
X₂ is CR₂;
R₁ is lower haloalkyl or halogen;
R₂, R₃, and R₄ are hydrogen;
R₇ is aryl or aryloxy; and
R₁₁, R₁₂, R₁₃, and R₁₄ are hydrogen.

7. The compound according to claim 6 that is
3′-chloro-N-(4-phenoxyphenyl)-3,6-dihydro-2H-1,2′-bipyridine-4-carboxamide; or
N-1,1′-biphenyl-4-yl-3′-chloro-3,6-dihydro-2H-1,2′-bipyridine-4-carboxamide.

8. The compound according to claim 2 wherein
- - - is a single bond;
X₁ is CR₁;
X₂ is CR₂;
R₁ is lower haloalkyl or halogen;
R₂, R₃, and R₄ are hydrogen;
R₇ is heterocycle; and
R₁₁, R₁₂, R₁₃, and R₁₄ are hydrogen.

9. The compound according to claim 8 that is
3′-chloro-N-[4-(1-piperidinyl)phenyl]-3,6-dihydro-2H-1,2′-bipyridine-4-carboxamide;
3′-chloro-N-[4-(4-morpholinyl)phenyl]-3,6-dihydro-2H-1,2′-bipyridine-4-carboxamide; or
N-[4-(1-azepanyl)phenyl]-3′-chloro-3,6-dihydro-2H-1,2′-bipyridine-4-carboxamide.

10. The compound according to claim 2 wherein
- - - is a single bond;
X₁ is CR₁;
X₂ is CR₂;
R₁ is lower haloalkyl or halogen;
R₂, R₃, and R₄ are hydrogen;
R₆ is lower alkyl, lower haloalkyl, or halogen; and
R₇, R₈, R₁₁, R₁₂, R₁₃, and R₁₄ are hydrogen.

11. The compound according to claim 10 that is
N-(3-tert-butylphenyl)-3′-chloro-3,6-dihydro-2H-1,2′-bipyridine-4-carboxamide; or
3′-chloro-N-(3-fluorophenyl)-3,6-dihydro-2H-1,2′-bipyridine-4-carboxamide.

12. The compound according to claim 2 wherein
- - - is absent;
X₁ is CR₁;
X₂ is CR₂;
R₁ is lower haloalkyl or halogen;
R₂, R₃, and R₄ are hydrogen;
R₇ is alkoxy, alkyl, alkylthio, haloalkoxy, haloalkyl, halogen, or —NR_{C}R_{D};
R₁ is hydrogen or hydroxy;
R₁₂, R₁₃, and R₁₄ are hydrogen; and
R_{C} and R_{D} are independently hydrogen or alkyl.

13. The compound according to claim 12 that is
N-(4-tert-butylphenyl)-1-(3-chloro-2-pyridinyl)-(cis)-3-hydroxy-4-piperidinecarboxamide;
N-(4-tert-butylphenyl)-1-(3-chloro-2-pyridinyl)-(trans)-3-hydroxy-4-piperidinecarboxamide; or 1-(3-chloro-2-pyridinyl)-4-hydroxy-N-[4-(trifluoromethyl)phenyl]-4-piperidinecarboxamide.

14. The compound according to claim 2 wherein
- - - is a single bond;
X₁ is N;
X₂ is CR₂;
R₂, R₃, and R₄ are hydrogen;
R₇ is alkoxy, alkyl, alkylthio, haloalkoxy, haloalkyl, haloalkylsulfonyl, haloalkylthio, halogen, or —NR_{C}R_{D};
R₁₁, R₁₂, R₁₃, and R₁₄ are hydrogen; and
R_{C} and R_{D} are independently hydrogen or alkyl.

15. The compound according to claim 12 that is
N-(4-chlorophenyl)-1-pyrimidin-2-yl-1,2,3,6-tetrahydropyridine-4-carboxamide;
1-pyrimidin-2-yl-N-{4-[(trifluoromethyl)thio]phenyl}-1,2,3,6-tetrahydropyridine-4-carboxamide; or
1-pyrimidin-2-yl-N-{4-[(trifluoromethyl)sulfonyl]phenyl}-1,2,3,6-tetrahydropyridine-4-carboxamide.

16. The compound according to claim 1 of formula (III) 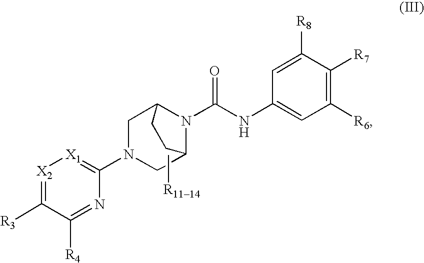
or a pharmaceutically acceptable salt or prodrug thereof.

17. The compound according to claim 16 wherein
X₁ is CR₁;
X₂ is CR₂;
R₁ is lower haloalkyl or halogen;
R₂, R₃, and R₄ are hydrogen;
R₇ is alkoxy, alkyl, alkylthio, haloalkoxy, haloalkyl, haloalkylthio, halogen, or —NR_{C}R_{D};
R₁₁, R₁₂, R₁₃, and R₁₄ are hydrogen; and
R_{C} and R_{D} are independently hydrogen or alkyl.

18. The compound according to claim 17 that is
N-(4-tert-butylphenyl)-3-(3-chloro-2-pyridinyl)-3,8-diazabicyclo[3.2.1]octane-8-carboxamide;
3-(3-chloro-2-pyridinyl)-N-(3,4-dichlorophenyl)-3,8-diazabicyclo[3.2.1]octane-8-carboxamide;
3-(3-chloro-2-pyridinyl)-N-[4-(trifluoromethyl)phenyl]-3,8-diazabicyclo[3.2.1]octane-8-carboxamide;
3-(3-chloro-2-pyridinyl)-N-(4-fluorophenyl)-3,8-diazabicyclo[3.2.1]octane-8-carboxamide;
N-(4-chlorophenyl)-3-(3-chloro-2-pyridinyl)-3,8-diazabicyclo[3.2.1]octane-8-carboxamide;
N-(4-bromophenyl)-3-(3-chloro-2-pyridinyl)-3,8-diazabicyclo[3.2.1]octane-8-carboxamide;
3-(3-chloro-2-pyridinyl)-N-(4-iodophenyl)-3,8-diazabicyclo[3.2.1]octane-8-carboxamide;
N-(4-butylphenyl)-3-(3-chloro-2-pyridinyl)-3,8-diazabicyclo[3.2.1]octane-8-carboxamide;
3-(3-chloro-2-pyridinyl)-N-(4-isopropylphenyl)-3,8-diazabicyclo[3.2.1]octane-8-carboxamide; or
3-(3-chloro-2-pyridinyl)-N-{⁴-[(trifluoromethyl)thio]phenyl}-3,8-diazabicyclo[3.2.1]octane-8-carboxamide.

19. The compound according to claim 16 wherein
X₁ is CR₁;
X₂ is CR₂;
R₁ is lower haloalkyl or halogen;
R₂, R₃, and R₄ are hydrogen;
R₆ is lower alkyl, lower haloalkyl, or halogen; and
R₇, R₈, R₁₁, R₁₂, R₁₃, and R₁₄ are hydrogen.

20. The compound according to claim 19 that is 3-(3-chloro-2-pyridinyl)-N-[3-(trifluoromethyl)phenyl]-3,8-diazabicyclo[3.2.1]octane-8-carboxamide.

21. The compound according to claim 1 of formula (IV) 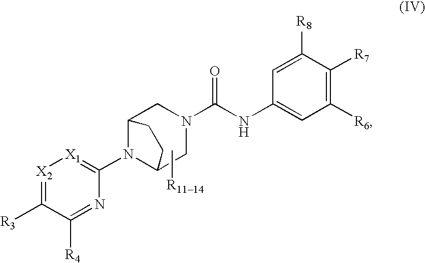
or a pharmaceutically acceptable salt or prodrug thereof.

22. The compound according to claim 21 wherein
X₁ is CR₁;
X₂ is CR₂;
R₁ is lower alkyl or halogen;
R₂, R₃, and R₄ are hydrogen;
R₇ is alkoxy, alkyl, alkylthio, haloalkoxy, haloalkyl, haloalkylthio, halogen, or —NR_{C}R_{D};
R₁₁, R₁₂, R₁₃, and R₁₄ are hydrogen; and
R_{C} and R_{D} are independently hydrogen or alkyl.

23. The compound according to claim 22 that is
N-(4-tert-butylphenyl)-8-(3-chloro-2-pyridinyl)-3,8-diazabicyclo[3.2.1]octane-3-carboxamide; or
8-(3-chloro-2-pyridinyl)-N-[4-(trifluoromethyl)phenyl]-3,8-diazabicyclo[3.2.1]octane-3-carboxamide.

24. A pharmaceutical composition comprising a therapeutically effective amount of a compound of formula (I-IV) or a pharmaceutically acceptable salt thereof.

25. A method of treating pain in a mammal, comprising administering a therapeutically effective amount of a compound of formula (I-IV) or a pharmaceutically acceptable salt thereof.
